(19) United States
(12) Patent Application Publication
(10) Pub. No.: US 2025/0302946 A1
(43) Pub. Date: Oct. 2, 2025

(54) COMPOSITIONS AND METHODS OF USE THEREOF

(71) Applicant: Seattle Project Corp., Dover, DE (US)

(72) Inventors: Sashikanth Banappagari, San Ramon, CA (US); Arunendra Saha Ray, Dublin, CA (US)

(21) Appl. No.: 19/223,664

(22) Filed: May 30, 2025

Related U.S. Application Data
(63) Continuation of application No. PCT/US2023/082034, filed on Dec. 1, 2023.
(60) Provisional application No. 63/429,897, filed on Dec. 2, 2022.

(51) Int. Cl. A61K 39/385; A61K 39/00; A61K 39/395; A61K 47/18; A61K 47/26

(52) U.S. Cl. CPC A61K 39/385; A61K 39/3955; A61K 47/18; A61K 47/183; A61K 47/26; A61K 2039/5256; A61K 2039/53; A61K 2039/54; A61K 2039/545

(57) ABSTRACT

Disclosed herein are a pharmaceutical composition comprising an RNA-based expression system suitable for storage and methods of making and using the same.

### SEQUENCE LISTING

The instant application contains a Sequence Listing which has been submitted electronically and is hereby incorporated herein by reference in its entirety. Said .XML copy, created on Mar. 8, 2024, is named GSO-117WO and is 12,288 bytes in size.

### BACKGROUND

Alphaviruses are a group of small positive-sense single-stranded RNA viruses that are responsible for many diseases in humans and other animals. See, e.g., “The Alphaviruses: Gene Expression, Replication and Evolution,” Microbiological Reviews, September 1994, p. 491-562; Jose et al., “A structural and functional perspective of alphavirus replication and assembly,” Future Micriobol., 2009, v.4:837-856. Because of their high replication efficiency and specificity, alphaviruses have proven to be useful in the engineering of self-replicating RNA vectors for the expression of heterologous proteins in mammalian cells. See, e.g., Frolov et al., “Alphavirus-based expression vectors: strategies and applications”, PNAS, 1996, v. 93, pp. 11371-11377; Young Kim, et al., “Enhancement of protein expression by alphavirus replicons by designing self-replicating subgenomic RNAs”, PNAS, 2014, v.11:29, pp. 10708-10713. Stability of these RNA-based expression systems can degrade overtime. Additionally, ease of storage is often hindered by the need to keep these expression systems at impractical low temperatures. Accordingly, there remains a need to develop pharmaceutical compositions that comprise these vector systems that are stable when stored.

### SUMMARY

Disclosed herein are pharmaceutical compositions comprising a lipid nanoparticle (LNP)-encapsulated self-amplifying alphavirus-based expression system or comprising a viral based expression system, further comprising a buffer system and two or more stabilizing excipients. Additionally, the present disclosure includes methods of inducing an immune response in a subject by administering a pharmaceutical composition to the subject.

### BRIEF DESCRIPTION OF THE DRAWINGS

FIG. 1A shows 6 line graphs illustrating concentration profile of DP in various formulations as described in Example 2.

FIG. 1B shows 6 line graphs illustrating % encapsulation profile of DP in various formulations as described in Example 2.

FIG. 2A shows 6 line graphs illustrating size profile of DP in various formulations as described in Example 2.

FIG. 2B shows 6 line graphs illustrating polydispersity of DP in various formulations as described in Example 2.

FIG. 3 shows 6 line graphs illustrating potency profile of DP in various formulations as described in Example 2.

FIG. 4 shows a bar graph illustrating full length profile of DP in various formulations as described in Example 2.

FIG. 5 shows 5 line graphs illustrating size profiles of samRNA-LNP in various formulations as described in Example 3.

FIG. 6 shows 5 graphs illustrating polydispersity profile of samRNA-LNP in various formulations as described in Example 3.

FIG. 7 shows 5 line graphs illustrating concentration profile of samRNA-LNP in various formulations as described in Example 3.

FIG. 8 shows 5 line graphs illustrating full length profile of samRNA-LNP in various formulations as described in Example 3.

FIG. 9 shows 5 graphs illustrating full length profile of a formulation as described in Example 3.

FIG. 10 shows 6 graphs illustrating potency profile of samRNA-LNP in various formulations as described in Example 3.

### DETAILED DESCRIPTION

Provided for herein are formulations of RNA-based expression systems. Such formulations increase the stability of an RNA-based expression system when stored for lengths of time at certain temperatures as described herein as compared to other formulated or unformulated RNA-based expression systems.

An RNA-based expression system may comprise one or more RNA constructs encapsulated in a lipid nanoparticle (LNP). The present disclosure includes a diversity of RNA-based expression systems. For example, an RNA-based expression system may be a messenger RNA (mRNA)-based expression system, circular (circRNA)-based expression system, single guide RNA (sgRNA)-based expression system, or self-amplifying RNA (samRNA) expression system. In some embodiments, an RNA-based expression system is a messenger RNA (mRNA)-based expression system. In some embodiments, an RNA-based expression system is a circular (circRNA)-based expression system. In some embodiments, an RNA-based expression system is a single guide RNA (sgRNA)-based expression system. In some embodiments, an RNA-based expression system is a self-amplifying RNA (samRNA) expression system.

The present disclosure includes an RNA-based expression system, further comprising a buffer, an amino acid, and a cryoprotectant. In some embodiments, a formulation provided herein comprises an RNA-based expression system and a buffer. In some embodiments, a formulation provided herein comprises an RNA-based expression system and a buffer. In some embodiments, a formulation provided herein comprises an RNA-based expression system and an amino acid. In some embodiments, a formulation provided herein comprises an RNA-based expression system and a cryoprotectant.

Stability of an RNA-based expression system and/or pharmaceutical composition contemplated herein can be determined by assessing change in one or more properties of the RNA-based expression system and/or pharmaceutical composition over time. For example, stability of an RNA-based expression system and/or pharmaceutical composition can be assessed by one or more assays comprising particle size, PDI, samRNA concentration, percent encapsulation, Full Length Profile (FLP) of samRNA and potency. In some embodiment, stability of an RNA-based expression system and/or pharmaceutical composition is assessed by evaluating particle size. In some embodiment, stability of an RNA-based expression system and/or pharmaceutical composition is assessed by evaluating PDI. In some embodiment, stability of an RNA-based expression system and/or pharmaceutical composition is assessed by evaluating samRNA concentration. In some embodiment, stability of an RNA-based expression system and/or pharmaceutical composition is assessed by evaluating FLP. In some embodiment, stability of an RNA-based expression system and/or pharmaceutical composition is assessed by evaluating percent encapsulation. In some embodiment, stability of an RNA-based expression system and/or pharmaceutical composition is assessed by evaluating potency.

Formulations provided here in enable stability of an RNA-based expression and/or pharmaceutical composition is determined after storage for a period of time as described herein. For example, stability of an RNA-based expression and/or pharmaceutical composition is evaluated after being stored for at least 1 day. In some embodiments, stability of an RNA-based expression and/or pharmaceutical composition is evaluated after being stored for at least 2 days. In some embodiments, stability of an RNA-based expression and/or pharmaceutical composition is evaluated after being stored for at least 3 days. In some embodiments, stability of an RNA-based expression and/or pharmaceutical composition is evaluated after being stored for at least 4 days. In some embodiments, stability of an RNA-based expression and/or pharmaceutical composition is evaluated after being stored for at least 5 days. In some embodiments, stability of an RNA-based expression and/or pharmaceutical composition is evaluated after being stored for at least 6 days. In some embodiments, stability of an RNA-based expression and/or pharmaceutical composition is evaluated after being stored for at least 1 week. In some embodiments, stability of an RNA-based expression and/or pharmaceutical composition is evaluated after being stored for at least 2 weeks. In some embodiments, stability of an RNA-based expression and/or pharmaceutical composition is evaluated after being stored for at least 3 weeks. In some embodiments, stability of an RNA-based expression and/or pharmaceutical composition is evaluated after being stored for at least one month. In some embodiments, stability of an RNA-based expression and/or pharmaceutical composition is evaluated after being stored for at least 2 months. In some embodiments, stability of an RNA-based expression and/or pharmaceutical composition is evaluated after being stored for at least 3 months. In some embodiments, stability of an RNA-based expression and/or pharmaceutical composition is evaluated after being stored for at least 4 months. In some embodiments, stability of an RNA-based expression and/or pharmaceutical composition is evaluated after being stored for at least 5 months. In some embodiments, stability of an RNA-based expression and/or pharmaceutical composition is evaluated after being stored for at least 6 months.

Formulations provided herein enable stability of an RNA-based expression at temperatures described throughout the present disclosure. For example, a formulation comprising an RNA-based expression as described herein may be stored at a temperature between −78° C. and 25° C. In some embodiments, a pharmaceutical composition comprising an RNA-based expression is stored at a temperature between −30° C. and −10° C. In some embodiments, a pharmaceutical composition comprising an RNA-based expression is stored at a temperature between −5° C. and 15° C. In some embodiments, a pharmaceutical composition comprising an RNA-based expression is stored at a temperature between 0° C. and 10° C. In some embodiments, a pharmaceutical composition comprising an RNA-based expression is stored at a temperature between 15° C. and 35° C. In some embodiments, a pharmaceutical composition comprising an RNA-based expression is stored at a temperature between 25° C. and 35° C. In some embodiments, a pharmaceutical composition comprising an RNA-based expression is stored at about −20° C. In some embodiments, a pharmaceutical composition comprising an RNA-based expression is stored at about 5° C. In some embodiments, a pharmaceutical composition comprising an RNA-based expression is stored at about 25° C.

### Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

As used herein, the term “pharmaceutical composition” is used interchangeably with the term “formulation.”

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

As used herein the term “antigen” is a substance that stimulates an immune response. An antigen can be a neoantigen. An antigen can be a “shared antigen” that is an antigen found among a specific population, e.g., a specific population of cancer patients.

As used herein the term “neoantigen” is an antigen that has at least one alteration that makes it distinct from the corresponding wild-type antigen, e.g., via mutation in a tumor cell or post-translational modification specific to a tumor cell. A neoantigen can include a polypeptide sequence or a nucleotide sequence. A mutation can include a frameshift or non-frameshift indel, missense or nonsense substitution, splice site alteration, genomic rearrangement or gene fusion, or any genomic or expression alteration giving rise to a neoORF. A mutations can also include a splice variant. Post-translational modifications specific to a tumor cell can include aberrant phosphorylation. Post-translational modifications specific to a tumor cell can also include a proteasome-generated spliced antigen. See Liepe et al., A large fraction of HLA class I ligands are proteasome-generated spliced peptides; Science. 2016 Oct. 21; 354(6310):354-358. The subject can be identified for administration through the use of various diagnostic methods, e.g., patient selection methods described further below.

As used herein the term “tumor antigen” is an antigen present in a subject's tumor cell or tissue but not in the subject's corresponding normal cell or tissue or derived from a polypeptide known to or have been found to have altered expression in a tumor cell or cancerous tissue in comparison to a normal cell or tissue.

As used herein the term “antigen-based vaccine” is a vaccine composition based on one or more antigens, e.g., a plurality of antigens. The vaccines can be nucleotide-based (e.g., virally based, RNA based, or DNA based), protein-based (e.g., peptide based), or a combination thereof.

As used herein the term “candidate antigen” is a mutation or other aberration giving rise to a sequence that may represent an antigen.

As used herein the term “coding region” is the portion(s) of a gene that encode protein.

As used herein the term “coding mutation” is a mutation occurring in a coding region.

As used herein the term “ORF” means open reading frame.

As used herein the term “NEO-ORF” is a tumor-specific ORF arising from a mutation or other aberration such as splicing.

As used herein the term “missense mutation” is a mutation causing a substitution from one amino acid to another.

As used herein the term “nonsense mutation” is a mutation causing a substitution from an amino acid to a stop codon or causing removal of a canonical start codon.

As used herein the term “frameshift mutation” is a mutation causing a change in the frame of the protein.

As used herein the term “indel” is an insertion or deletion of one or more nucleic acids.

As used herein, the term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Alternatively, sequence similarity or dissimilarity can be established by the combined presence or absence of particular nucleotides, or, for translated sequences, amino acids at selected sequence positions (e.g., sequence motifs).

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information.

As used herein the term “non-stop or read-through” is a mutation causing the removal of the natural stop codon.

As used herein the term “epitope” is the specific portion of an antigen typically bound by an antibody or T cell receptor.

As used herein the term “immunogenic” is the ability to stimulate an immune response, e.g., via T cells, B cells, or both.

As used herein the term “HLA binding affinity” “MHC binding affinity” means affinity of binding between a specific antigen and a specific MHC allele.

As used herein the term “bait” is a nucleic acid probe used to enrich a specific sequence of DNA or RNA from a sample.

As used herein the term “variant” is a difference between a subject's nucleic acids and the reference human genome used as a control.

As used herein the term “variant call” is an algorithmic determination of the presence of a variant, typically from sequencing.

As used herein the term “polymorphism” is a germline variant, i.e., a variant found in all DNA-bearing cells of an individual.

As used herein the term “somatic variant” is a variant arising in non-germline cells of an individual.

As used herein the term “allele” is a version of a gene or a version of a genetic sequence or a version of a protein.

As used herein the term “HLA type” is the complement of HLA gene alleles.

As used herein the term “nonsense-mediated decay” or “NMD” is a degradation of an mRNA by a cell due to a premature stop codon.

As used herein the term “truncal mutation” is a mutation originating early in the development of a tumor and present in a substantial portion of the tumor's cells.

As used herein the term “subclonal mutation” is a mutation originating later in the development of a tumor and present in only a subset of the tumor's cells.

As used herein the term “exome” is a subset of the genome that codes for proteins. An exome can be the collective exons of a genome.

As used herein the term “logistic regression” is a regression model for binary data from statistics where the logit of the probability that the dependent variable is equal to one is modeled as a linear function of the dependent variables.

As used herein the term “neural network” is a machine learning model for classification or regression consisting of multiple layers of linear transformations followed by element-wise nonlinearities typically trained via stochastic gradient descent and back-propagation.

As used herein the term “proteome” is the set of all proteins expressed and/or translated by a cell, group of cells, or individual.

As used herein the term “peptidome” is the set of all peptides presented by MHC-I or MHC-II on the cell surface. The peptidome may refer to a property of a cell or a collection of cells (e.g., the tumor peptidome, meaning the union of the peptidomes of all cells that comprise the tumor, or the infectious disease peptidome, meaning the union of the peptidomes of all cells that are infected by the infectious disease).

As used herein the term “ELISPOT” means Enzyme-linked immunosorbent spot assay—which is a common method for monitoring immune responses in humans and animals.

As used herein the term “dextramers” is a dextran-based peptide-MHC multimers used for antigen-specific T-cell staining in flow cytometry.

As used herein the term “tolerance or immune tolerance” is a state of immune non-responsiveness to one or more antigens, e.g., self-antigens.

As used herein the term “central tolerance” is a tolerance affected in the thymus, either by deleting self-reactive T-cell clones or by promoting self-reactive T-cell clones to differentiate into immunosuppressive regulatory T-cells (Tregs).

As used herein the term “peripheral tolerance” is a tolerance affected in the periphery by downregulating or energizing self-reactive T-cells that survive central tolerance or promoting these T cells to differentiate into Tregs.

The term “sample” can include a single cell or multiple cells or fragments of cells or an aliquot of body fluid, taken from a subject, by means including venipuncture, excretion, ejaculation, massage, biopsy, needle aspirate, lavage sample, scraping, surgical incision, or intervention or other means known in the art.

The term “subject” encompasses a cell, tissue, or organism, human or non-human, whether in vivo, ex vivo, or in vitro, male or female. The term subject is inclusive of mammals including humans.

The term “mammal” encompasses both humans and non-humans and includes but is not limited to humans, non-human primates, canines, felines, murines, bovines, equines, and porcines.

The term “clinical factor” refers to a measure of a condition of a subject, e.g., disease activity or severity. “Clinical factor” encompasses all markers of a subject's health status, including non-sample markers, and/or other characteristics of a subject, such as, without limitation, age and gender. A clinical factor can be a score, a value, or a set of values that can be obtained from evaluation of a sample (or population of samples) from a subject or a subject under a determined condition. A clinical factor can also be predicted by markers and/or other parameters such as gene expression surrogates. Clinical factors can include tumor type, tumor sub-type, infection type, infection sub-type, and smoking history.

The term “antigen-encoding nucleic acid sequences derived from a tumor” refers to nucleic acid sequences obtained from the tumor, e.g. via RT-PCR; or sequence data obtained by sequencing the tumor and then synthesizing the nucleic acid sequences using the sequencing data, e.g., via various synthetic or PCR-based methods known in the art. Derived sequences can include nucleic acid sequence variants, such as sequence-optimized nucleic acid sequence variants (e.g., codon-optimized and/or otherwise optimized for expression), that encode the same polypeptide sequence as the corresponding native nucleic acid sequence obtained from a tumor.

The term “antigen-encoding nucleic acid sequences derived from an infection” refers to nucleic acid sequences obtained from infected cells or an infectious disease organism, e.g. via RT-PCR; or sequence data obtained by sequencing the infected cell or infectious disease organism and then synthesizing the nucleic acid sequences using the sequencing data, e.g., via various synthetic or PCR-based methods known in the art. Derived sequences can include nucleic acid sequence variants, such as sequence-optimized nucleic acid sequence variants (e.g., codon-optimized and/or otherwise optimized for expression), that encode the same polypeptide sequence as the corresponding native infectious disease organism nucleic acid sequence. Derived sequences can include nucleic acid sequence variants that encode a modified infectious disease organism polypeptide sequence having one or more (e.g., 1, 2, 3, 4, or 5) mutations relative to a native infectious disease organism polypeptide sequence. For example, a modified polypeptide sequence can have one or more missense mutations relative to the native polypeptide sequence of an infectious disease organism protein.

The term “alphavirus” refers to members of the family Togaviridae and are positive-sense single-stranded RNA viruses. Alphaviruses are typically classified as either Old World, such as Sindbis, Ross River, Mayaro, Chikungunya, and Semliki Forest viruses, or New World, such as eastern equine encephalitis, Aura, Fort Morgan, or Venezuelan equine encephalitis and its derivative strain TC-83. Alphaviruses are typically self-replicating RNA viruses.

The term “alphavirus backbone” refers to minimal sequence(s) of an alphavirus that allow for self-replication of the viral genome. Minimal sequences can include conserved sequences for nonstructural protein-mediated amplification, a nonstructural protein 1 (nsP1) gene, a nsP2 gene, a nsP3 gene, a nsP4 gene, and a polyA sequence, as well as sequences for expression of subgenomic viral RNA including a subgenomic (e.g., a 26S) promoter element.

The term “sequences for nonstructural protein-mediated amplification” includes alphavirus conserved sequence elements (CSE) well known to those in the art. CSEs include, but are not limited to, an alphavirus 5′ UTR, a 51-nt CSE, a 24-nt CSE, a subgenomic promoter sequence (e.g., a 26S subgenomic promoter sequence), a 19-nt CSE, and an alphavirus 3′ UTR.

The term “RNA polymerase” includes polymerases that catalyze the production of RNA polynucleotides from a DNA template. RNA polymerases include, but are not limited to, bacteriophage derived polymerases including T3, T7, and SP6.

The term “lipid” includes hydrophobic and/or amphiphilic molecules. Lipids can be cationic, anionic, or neutral. Lipids can be synthetic or naturally derived, and in some instances biodegradable. Lipids can include cholesterol, phospholipids, lipid conjugates including, but not limited to, polyethyleneglycol (PEG) conjugates (PEGylated lipids), waxes, oils, glycerides, fats, and fat-soluble vitamins. Lipids can also include dilinoleylmethyl-4-dimethylaminobutyrate (MC3) and MC3-like molecules.

The term “lipid nanoparticle” or “LNP” includes vesicle like structures formed using a lipid containing membrane surrounding an aqueous interior, also referred to as liposomes. Lipid nanoparticles includes lipid-based compositions with a solid lipid core stabilized by a surfactant. The core lipids can be fatty acids, acylglycerols, waxes, and mixtures of these surfactants. Biological membrane lipids such as phospholipids, sphingomyelins, bile salts (sodium taurocholate), and sterols (cholesterol) can be utilized as stabilizers. Lipid nanoparticles can be formed using defined ratios of different lipid molecules, including, but not limited to, defined ratios of one or more cationic, anionic, or neutral lipids. Lipid nanoparticles can encapsulate molecules within an outer-membrane shell and subsequently can be contacted with target cells to deliver the encapsulated molecules to the host cell cytosol. Lipid nanoparticles can be modified or functionalized with non-lipid molecules, including on their surface. Lipid nanoparticles can be single-layered (unilamellar) or multi-layered (multilamellar). Lipid nanoparticles can be complexed with nucleic acid. Unilamellar lipid nanoparticles can be complexed with nucleic acid, wherein the nucleic acid is in the aqueous interior. Multilamellar lipid nanoparticles can be complexed with nucleic acid, wherein the nucleic acid is in the aqueous interior, or to form or sandwiched between

Abbreviations: MHC: major histocompatibility complex; HLA: human leukocyte antigen, or the human MHC gene locus; NGS: next-generation sequencing; PPV: positive predictive value; TSNA: tumor-specific neoantigen; FFPE: formalin-fixed, paraffin-embedded; NMD: nonsense-mediated decay; NSCLC: non-small-cell lung cancer; DC: dendritic cell.

It should be noted that, as used in the specification and the appended claims, the singular forms “a,” “an,” and “the” include plural referents unless the context clearly dictates otherwise.

Unless specifically stated or otherwise apparent from context, as used herein the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any terms not directly defined herein shall be understood to have the meanings commonly associated with them as understood within the art of the invention. Certain terms are discussed herein to provide additional guidance to the practitioner in describing the compositions, devices, methods and the like of aspects, of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects, of the invention herein.

All references, issued patents, and patent applications cited within the body of the specification are hereby incorporated by reference in their entirety, for all purposes.

### Antigens

Antigens can include nucleotides or polypeptides. For example, an antigen can be an RNA sequence that encodes for a polypeptide sequence. Antigens useful in vaccines can therefore include nucleotide sequences or polypeptide sequences.

Disclosed herein are isolated peptides that comprise tumor specific mutations identified by the methods disclosed herein, peptides that comprise known tumor specific mutations, and mutant polypeptides or fragments thereof identified by methods disclosed herein. Neoantigen peptides can be described in the context of their coding sequence where a neoantigen includes the nucleotide sequence (e.g., DNA or RNA) that codes for the related polypeptide sequence.

Also disclosed herein are peptides derived from any polypeptide known to or have been found to have altered expression in a tumor cell or cancerous tissue in comparison to a normal cell or tissue, for example any polypeptide known to or have been found to be aberrantly expressed in a tumor cell or cancerous tissue in comparison to a normal cell or tissue. Suitable polypeptides from which the antigenic peptides can be derived can be found for example in the COSMIC database. COSMIC curates comprehensive information on somatic mutations in human cancer. The peptide contains the tumor specific mutation.

Also disclosed herein are peptides derived from any polypeptide associated with an infectious disease organism, an infection in a subject, or an infected cell of a subject. Antigens can be derived from nucleotide sequences or polypeptide sequences of an infectious disease organism. Polypeptide sequences of an infectious disease organism include, but are not limited to, a pathogen-derived peptide, a virus-derived peptide, a bacteria-derived peptide, a fungus-derived peptide, and/or a parasite-derived peptide. Infectious disease organism include, but are not limited to, Severe acute respiratory syndrome-related coronavirus (SARS), severe acute respiratory syndrome coronavirus 2 (SARS-CoV-2), Ebola, HIV, Hepatitis B virus (HBV), influenza, Hepatitis C virus (HCV), Human papillomavirus (HPV), Cytomegalovirus (CMV), Chikungunya virus, Respiratory syncytial virus (RSV), Dengue virus, an Orthomyxoviridae family virus, and tuberculosis.

Antigens can be selected that are predicted to be presented on the cell surface of a cell, such as a tumor cell, an infected cell, or an immune cell, including professional antigen presenting cells such as dendritic cells. Antigens can be selected that are predicted to be immunogenic.

One or more polypeptides encoded by an antigen nucleotide sequence can comprise at least one of: a binding affinity with MHC with an IC₅₀ value of less than 1000 nM, for MHC Class I peptides a length of 8-15, 8, 9, 10, 11, 12, 13, 14, or 15 amino acids, presence of sequence motifs within or near the peptide promoting proteasome cleavage, and presence or sequence motifs promoting TAP transport. For MHC Class II peptides a length 6-30, 6, 7, 8, 9, 10, 11, 12, 13, 14, 15, 16, 17, 18, 19, 20, 21, 22, 23, 24, 25, 26, 27, 28, 29, or 30 amino acids, presence of sequence motifs within or near the peptide promoting cleavage by extracellular or lysosomal proteases (e.g., cathepsins) or HLA-DM catalyzed HLA binding.

One or more antigens can be presented on the surface of a tumor. One or more antigens can be presented on the surface of an infected cell.

One or more antigens can be immunogenic in a subject having a tumor, e.g., capable of eliciting a T cell response or a B cell response in the subject. One or more antigens can be immunogenic in a subject having or suspected to have an infection, e.g., capable of eliciting a T cell response or a B cell response in the subject. One or more antigens can be immunogenic in a subject at risk of an infection, e.g., capable of eliciting a T cell response or a B cell response in the subject that provides immunological protection (i.e., immunity) against the infection, e.g., such as stimulating the production of memory T cells, memory B cells, or antibodies specific to the infection.

One or more antigens can be capable of eliciting a B cell response, such as the production of antibodies that recognize the one or more antigens. Antibodies can recognize linear polypeptide sequences or recognize secondary and tertiary structures. Accordingly, B cell antigens can include linear polypeptide sequences or polypeptides having secondary and tertiary structures, including, but not limited to, full-length proteins, protein subunits, protein domains, or any polypeptide sequence known or predicted to have secondary and tertiary structures.

One or more antigens that induce an autoimmune response in a subject can be excluded from consideration in the context of vaccine generation for a subject.

The size of at least one antigenic peptide molecule (e.g., an epitope sequence) can comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120 or greater amino molecule residues, and any range derivable therein. In specific embodiments the antigenic peptide molecules are equal to or less than 50 amino acids.

Antigenic peptides and polypeptides can be for MHC Class I 15 residues or less in length and usually consist of between about 8 and about 11 residues, particularly 9 or 10 residues; for MHC Class II, 6-30 residues, inclusive.

If desirable, a longer peptide can be designed in several ways. In one case, when presentation likelihoods of peptides on HLA alleles are predicted or known, a longer peptide could consist of either: (1) individual presented peptides with an extension of 2-5 amino acids toward the N- and C-terminus of each corresponding gene product; (2) a concatenation of some or all of the presented peptides with extended sequences for each. In another case, when sequencing reveals a long (>10 residues) neoepitope sequence present in the tumor (e.g., due to a frameshift, read-through or intron inclusion that leads to a novel peptide sequence), a longer peptide would consist of: (3) the entire stretch of novel tumor-specific or infectious disease-specific amino acids—thus bypassing the need for computational or in vitro test-based selection of the strongest HLA-presented shorter peptide. In both cases, use of a longer peptide allows endogenous processing by patient cells and may lead to more effective antigen presentation and induction of T cell responses.

Antigenic peptides and polypeptides can be presented on an HLA protein. In some aspects, antigenic peptides and polypeptides are presented on an HLA protein with greater affinity than a wild-type peptide. In some aspects, an antigenic peptide or polypeptide can have an IC50 of at least less than 5000 nM, at least less than 1000 nM, at least less than 500 nM, at least less than 250 nM, at least less than 200 nM, at least less than 150 nM, at least less than 100 nM, at least less than 50 nM or less.

In some aspects, antigenic peptides and polypeptides do not induce an autoimmune response and/or invoke immunological tolerance when administered to a subject.

Also provided are compositions comprising at least two or more antigenic peptides. In some embodiments the composition contains at least two distinct peptides. At least two distinct peptides can be derived from the same polypeptide. By distinct polypeptides is meant that the peptide varies by length, amino acid sequence, or both. The peptides can be derived from any polypeptide known to or have been found to contain a tumor specific mutation or peptides derived from any polypeptide known to or have been found to have altered expression in a tumor cell or cancerous tissue in comparison to a normal cell or tissue, for example any polypeptide known to or have been found to be aberrantly expressed in a tumor cell or cancerous tissue in comparison to a normal cell or tissue. The peptides can be derived from any polypeptide known to or suspected to be associated with an infectious disease organism, or peptides derived from any polypeptide known to or have been found to have altered expression in an infected cell in comparison to a normal cell or tissue (e.g., an infectious disease polynucleotide or polypeptide, including infectious disease polynucleotides or polypeptides with expression restricted to a host cell). Suitable polypeptides from which the antigenic peptides can be derived can be found for example in the COSMIC database or the AACR Genomics Evidence Neoplasia Information Exchange (GENIE) database. COSMIC curates comprehensive information on somatic mutations in human cancer. AACR GENIE aggregates and links clinical-grade cancer genomic data with clinical outcomes from tens of thousands of cancer patients. In some aspects, the tumor specific mutation is a driver mutation for a particular cancer type.

Antigenic peptides and polypeptides having a desired activity or property can be modified to provide certain desired attributes, e.g., improved pharmacological characteristics, while increasing or at least retaining substantially all of the biological activity of the unmodified peptide to bind the desired MHC molecule and activate the appropriate T cell. For instance, antigenic peptide and polypeptides can be subject to various changes, such as substitutions, either conservative or non-conservative, where such changes might provide for certain advantages in their use, such as improved MHC binding, stability or presentation. By conservative substitutions is meant replacing an amino acid residue with another which is biologically and/or chemically similar, e.g., one hydrophobic residue for another, or one polar residue for another. The substitutions include combinations such as Gly, Ala; Val, Ile, Leu, Met; Asp, Glu; Asn, Gln; Ser, Thr; Lys, Arg; and Phe, Tyr. The effect of single amino acid substitutions may also be probed using D-amino acids. Such modifications can be made using well known peptide synthesis procedures, as described in e.g., Merrifield, Science 232:341-347 (1986), Barany & Merrifield, The Peptides, Gross & Meienhofer, eds. (N.Y., Academic Press), pp. 1-284 (1979); and Stewart & Young, Solid Phase Peptide Synthesis, (Rockford, Ill., Pierce), 2d Ed. (1984).

Modifications of peptides and polypeptides with various amino acid mimetics or unnatural amino acids can be particularly useful in increasing the stability of the peptide and polypeptide in vivo. Stability can be assayed in a number of ways. For instance, peptidases and various biological media, such as human plasma and serum, have been used to test stability. See, e.g., Verhoef et al., Eur. J. Drug Metab Pharmacokin. 11:291-302 (1986). Half-life of the peptides can be conveniently determined using a 25% human serum (v/v) assay. The protocol is generally as follows. Pooled human serum (Type AB, non-heat inactivated) is delipidated by centrifugation before use. The serum is then diluted to 25% with RPMI tissue culture media and used to test peptide stability. At predetermined time intervals a small amount of reaction solution is removed and added to either 6% aqueous trichloroacetic acid or ethanol. The cloudy reaction sample is cooled (4 degrees C.) for 15 minutes and then spun to pellet the precipitated serum proteins. The presence of the peptides is then determined by reversed-phase HPLC using stability-specific chromatography conditions.

The peptides and polypeptides can be modified to provide desired attributes other than improved serum half-life. For instance, the ability of the peptides to induce CTL activity can be enhanced by linkage to a sequence which contains at least one epitope that is capable of inducing a T helper cell response. Immunogenic peptides/T helper conjugates can be linked by a spacer molecule. The spacer is typically comprised of relatively small, neutral molecules, such as amino acids or amino acid mimetics, which are substantially uncharged under physiological conditions. The spacers are typically selected from, e.g., Ala, Gly, or other neutral spacers of nonpolar amino acids or neutral polar amino acids. It will be understood that the optionally present spacer need not be comprised of the same residues and thus can be a hetero- or homo-oligomer. When present, the spacer will usually be at least one or two residues, more usually three to six residues. Alternatively, the peptide can be linked to the T helper peptide without a spacer.

An antigenic peptide can be linked to the T helper peptide either directly or via a spacer either at the amino or carboxy terminus of the peptide. The amino terminus of either the antigenic peptide or the T helper peptide can be acylated. Exemplary T helper peptides include tetanus toxoid 830-843, influenza 307-319, malaria circumsporozoite 382-398 and 378-389.

Proteins or peptides can be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and can be found at computerized databases known to those of ordinary skill in the art. One such database is the National Center for Biotechnology Information's Genbank and GenPept databases located at the National Institutes of Health website. The coding regions for known genes can be amplified and/or expressed using the techniques disclosed herein or as would be known to those of ordinary skill in the art. Alternatively, various commercial preparations of proteins, polypeptides and peptides are known to those of skill in the art.

In a further aspect an antigen includes a nucleic acid (e.g., polynucleotide) that encodes an antigenic peptide or portion thereof. The polynucleotide can be, e.g., DNA, cDNA, PNA, CNA, RNA (e.g., mRNA), either single- and/or double-stranded, or native or stabilized forms of polynucleotides, such as, e.g., polynucleotides with a phosphonothioate backbone, or combinations thereof and it may or may not contain introns. A still further aspect provides an expression vector capable of expressing a polypeptide or portion thereof. Expression vectors for different cell types are well known in the art and can be selected without undue experimentation. Generally, DNA is inserted into an expression vector, such as a plasmid, in proper orientation and correct reading frame for expression. If necessary, DNA can be linked to the appropriate transcriptional and translational regulatory control nucleotide sequences recognized by the desired host, although such controls are generally available in the expression vector. The vector is then introduced into the host through standard techniques. Guidance can be found e.g., in Sambrook et al. (1989) Molecular Cloning, A Laboratory Manual, Cold Spring Harbor Laboratory, Cold Spring Harbor, N.Y.

### Lipid Nanoparticles

In some aspects, any of the above compositions further comprise a nanoparticulate delivery vehicle. The nanoparticulate delivery vehicle, in some aspects, may be a lipid nanoparticle (LNP). In some aspects, the LNP comprises ionizable amino lipids. In some aspects, the ionizable amino lipids comprise MC3-like (dilinoleylmethyl-4-dimethylaminobutyrate) molecules. In some aspects, the nanoparticulate delivery vehicle encapsulates the neoantigen expression system.

In some aspects, any of the above compositions further comprise a plurality of LNPs, wherein the LNPs comprise: the neoantigen expression system; a cationic lipid; a non-cationic lipid; and a conjugated lipid that inhibits aggregation of the LNPs, wherein at least about 95% of the LNPs in the plurality of LNPs either: have a non-lamellar morphology; or are electron-dense.

In some aspects, the non-cationic lipid is a mixture of (1) a phospholipid and (2) cholesterol or a cholesterol derivative.

In some aspects, the conjugated lipid that inhibits aggregation of the LNPs is a polyethyleneglycol (PEG)-lipid conjugate. In some aspects, the PEG-lipid conjugate is selected from the group consisting of a PEG-diacylglycerol (PEG-DAG) conjugate, a PEG dialkyloxypropyl (PEG-DAA) conjugate, a PEG-phospholipid conjugate, a PEG-ceramide (PEG-Cer) conjugate, and a mixture thereof. In some aspects, the PEG-DAA conjugate is a member selected from the group consisting of a PEG-didecyloxypropyl (C₁₀) conjugate, a PEG-dilauryloxypropyl (C₁₂) conjugate, a PEG-dimyristyloxypropyl (C₁₄) conjugate, a PEG-dipalmityloxypropyl (C₁₆) conjugate, a PEG-distearyloxypropyl (C₁₈) conjugate, and a mixture thereof.

In some aspects, the neoantigen expression system is fully encapsulated in the LNPs.

In some aspects, the non-lamellar morphology of the LNPs comprises an inverse hexagonal (H_{II}) or cubic phase structure.

In some aspects, the cationic lipid comprises from about 10 mol % to about 50 mol % of the total lipid present in the LNPs. In some aspects, the cationic lipid comprises from about 20 mol % to about 50 mol % of the total lipid present in the LNPs. In some aspects, the cationic lipid comprises from about 20 mol % to about 40 mol % of the total lipid present in the LNPs.

In some aspects, the non-cationic lipid comprises from about 10 mol % to about 60 mol % of the total lipid present in the LNPs. In some aspects, the non-cationic lipid comprises from about 20 mol % to about 55 mol % of the total lipid present in the LNPs. In some aspects, the non-cationic lipid comprises from about 25 mol % to about 50 mol % of the total lipid present in the LNPs.

In some aspects, the conjugated lipid comprises from about 0.5 mol % to about 20 mol % of the total lipid present in the LNPs. In some aspects, the conjugated lipid comprises from about 2 mol % to about 20 mol % of the total lipid present in the LNPs. In some aspects, the conjugated lipid comprises from about 1.5 mol % to about 18 mol % of the total lipid present in the LNPs.

In some aspects, greater than 95% of the LNPs have a non-lamellar morphology. In some aspects, greater than 95% of the LNPs are electron dense.

In some aspects, any of the above compositions further comprise a plurality of LNPs, wherein the LNPs comprise: a cationic lipid comprising from 50 mol % to 65 mol % of the total lipid present in the LNPs; a conjugated lipid that inhibits aggregation of LNPs comprising from 0.5 mol % to 2 mol % of the total lipid present in the LNPs; and a non-cationic lipid comprising either: a mixture of a phospholipid and cholesterol or a derivative thereof, wherein the phospholipid comprises from 4 mol % to 10 mol % of the total lipid present in the LNPs and the cholesterol or derivative thereof comprises from 30 mol % to 40 mol % of the total lipid present in the LNPs; a mixture of a phospholipid and cholesterol or a derivative thereof, wherein the phospholipid comprises from 3 mol % to 15 mol % of the total lipid present in the LNPs and the cholesterol or derivative thereof comprises from 30 mol % to 40 mol % of the total lipid present in the LNPs; or up to 49.5 mol % of the total lipid present in the LNPs and comprising a mixture of a phospholipid and cholesterol or a derivative thereof, wherein the cholesterol or derivative thereof comprises from 30 mol % to 40 mol % of the total lipid present in the LNPs.

In some aspects, any of the above compositions further comprise a plurality of LNPs, wherein the LNPs comprise: a cationic lipid comprising from 50 mol % to 85 mol % of the total lipid present in the LNPs; a conjugated lipid that inhibits aggregation of LNPs comprising from 0.5 mol % to 2 mol % of the total lipid present in the LNPs; and a non-cationic lipid comprising from 13 mol % to 49.5 mol % of the total lipid present in the LNPs.

In some aspects, the phospholipid comprises dipalmitoylphosphatidylcholine (DPPC), distearoylphosphatidylcholine (DSPC), or a mixture thereof.

In some aspects, the conjugated lipid comprises a polyethyleneglycol (PEG)-lipid conjugate. In some aspects, the PEG-lipid conjugate comprises a PEG-diacylglycerol (PEG-DAG) conjugate, a PEG-dialkyloxypropyl (PEG-DAA) conjugate, or a mixture thereof. In some aspects, the PEG-DAA conjugate comprises a PEG-dimyristyloxypropyl (PEG-DMA) conjugate, a PEG-distearyloxypropyl (PEG-DSA) conjugate, or a mixture thereof. In some aspects, the PEG portion of the conjugate has an average molecular weight of about 2,000 Daltons.

In some aspects, the conjugated lipid comprises from 1 mol % to 2 mol % of the total lipid present in the LNPs.

In some aspects, the LNP comprises a compound having a structure of Formula I:

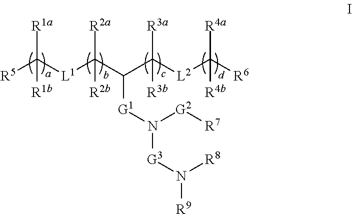

or a pharmaceutically acceptable salt, tautomer, prodrug or stereoisomer thereof, wherein: L¹ and L² are each independently —O(C═O)—, —(C═O)O—, —C(═O)—, —O—, —S(O)ₓ—, —S—S—, —C(═O)S—, —SC(═O)—, —R^{a}C(═O)—, —C(═O) R^{a}—, —R^{a}C(═O) R^{a}—, —OC(═O) R^{a}—, —R^{a}C(═O)O— or a direct bond; G¹ is C₁-C₂ alkylene, —(C═O)—, —O(C═O)—, —SC(═O)—, —R^{a}C(═O)— or a direct bond: —C(═O)—, —(C═O)O—, —C(═O)S—, —C(═O) R^{a}— or a direct bond; G is C₁-C₆ alkylene; R^{a} is H or C₁-C₁₂ alkyl; R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond; R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond; R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond; R^{4a} and R^{4b} are, at each occurrence, independently either: (a) H or C1-C12 alkyl; or (b) R^{4a} is H or C1-C12 alkyl, and R^{4b} together with the carbon atom to which it is bound is taken together with an adjacent R^{4b} and the carbon atom to which it is bound to form a carbon-carbon double bond; R⁵ and R⁶ are each independently H or methyl; R⁷ is C₄-C₂₀ alkyl; R⁸ and R⁹ are each independently C₁-C₁₂ alkyl; or R⁸ and R⁹, together with the nitrogen atom to which they are attached, form a 5, 6 or 7-membered heterocyclic ring; a, b, c and d are each independently an integer from 1 to 24; and x is 0, 1 or 2.

In some aspects, the LNP comprises a compound having a structure of Formula II:

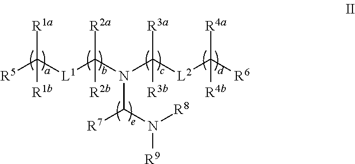

or a pharmaceutically acceptable salt, tautomer, prodrug or stereoisomer thereof, wherein: L¹ and L² are each independently —O(C═O)—, —(C═O)O— or a carbon-carbon double bond; R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond; R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond; R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond; R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond; R⁵ and R⁶ are each independently methyl or cycloalkyl; R⁷ is, at each occurrence, independently H or C₁-C₁₂ alkyl; R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom; a and d are each independently an integer from 0 to 24; b and c are each independently an integer from 1 to 24; and e is 1 or 2, provided that: at least one of R^{1a}, R^{2a}, R^{3a} or R^{4a} is C₁-C₁₂ alkyl, or at least one of L¹ or L² is —O(C═O)— or —(C═O)O—; and R^{1a} and R^{1b} are not isopropyl when a is 6 or n-butyl when a is 8.

In some aspects, any of the above compositions further comprise one or more excipients comprising a neutral lipid, a steroid, and a polymer conjugated lipid. In some aspects, the neutral lipid comprises at least one of 1,2-Distearoyl-sn-glycero-3-phosphocholine (DSPC), 1,2-Dipalmitoyl-sn-glycero-3-phosphocholine (DPPC), 1,2-Dimyristoyl-sn-glycero-3-phosphocholine (DMPC), 1-Palmitoyl-2-oleoyl-sn-glycero-3-phosphocholine (POPC), 1,2-dioleoyl-sn-glycero-3-phosphocholine (DOPC), and 1,2-Dioleoyl-sn-glycero-3-phosphoethanolamine (DOPE). In some aspects, the neutral lipid is DSPC.

In some aspects, the molar ratio of the compound to the neutral lipid ranges from about 2:1 to about 8:1.

In some aspects, the steroid is cholesterol. In some aspects, the molar ratio of the compound to cholesterol ranges from about 2:1 to 1:1.

In some aspects, the polymer conjugated lipid is a pegylated lipid. In some aspects, the molar ratio of the compound to the pegylated lipid ranges from about 100:1 to about 25:1. In some aspects, the pegylated lipid is PEG-DAG, a PEG polyethylene (PEG-PE), a PEG-succinyl-diacylglycerol (PEG-S-DAG), PEG-cer or a PEG dialkyoxypropylcarbamate. In some aspects, the pegylated lipid has the following structure III:

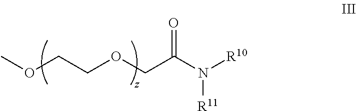

or a pharmaceutically acceptable salt, tautomer or stereoisomer thereof, wherein: R¹⁰ and R¹¹ are each independently a straight or branched, saturated or unsaturated alkyl chain containing from 10 to 30 carbon atoms, wherein the alkyl chain is optionally interrupted by one or more ester bonds; and z has a mean value ranging from 30 to 60. In some aspects, R¹⁰ and R¹¹ are each independently straight, saturated alkyl chains having 12 to 16 carbon atoms. In some aspects, the average z is about 45.

In some aspects, the LNP self-assembles into non-bilayer structures when mixed with polyanionic nucleic acid. In some aspects, the non-bilayer structures have a diameter between 60 nm and 120 nm. In some aspects, the non-bilayer structures have a diameter of about 70 nm, about 80 nm, about 90 nm, or about 100 nm. In some aspects, wherein the nanoparticulate delivery vehicle has a diameter of about 100 nm.

### Payloads and Antigens

A payload nucleic acid sequence can be any nucleic acid sequence desired to be delivered to a cell of interest. In general, the payload is a nucleic acid sequence linked to a promoter to drive expression of the nucleic acid sequence. The payload nucleic acid sequence can encode a polypeptide (i.e., a nucleic acid sequence capable of being transcribed and translated into a protein). In general, a payload nucleic acid sequence encoding a peptide can encode any protein desired to be expressed in a cell. Examples of proteins include, but are not limited to, an antigen (e.g., an MHC class I epitope, an MHC class II epitope, or an epitope capable of stimulating a B cell response), an antibody, a cytokine, a chimeric antigen receptor (CAR), a T-cell receptor, or a genome-editing system component (e.g., a nuclease used in a genome-editing system). Genome-editing systems include, but are not limited to, a CRISPR system, a zinc-finger system, a meganuclease system, or a TALEN system. The payload nucleic acid sequence can be non-coding (i.e., a nucleic acid sequence capable of being transcribed but is not translated into a protein). In general, a non-coding payload nucleic acid sequence can be any non-coding polynucleotide desired to be expressed in a cell. Examples of non-coding polynucleotides include, but are not limited to, RNA interference (RNAi) polynucleotides (e.g., antisense oligonucleotides, shRNAs, siRNAs, miRNAs etc.) or genome-editing system polynucleotide (e.g., a guide RNA [gRNA], a single-guide RNA [sgRNA], a trans-activating CRISPR [tracrRNA], and/or a CRISPR RNA [crRNA]). A payload nucleic acid sequence can encode two or more (e.g., 2, 3, 4, 5 or more) distinct polypeptides (e.g., two or more distinct epitope sequences linked together) or contain two or more distinct non-coding nucleic acid sequences (e.g., two or more distinct RNAi polynucleotides). A payload nucleic acid sequence can have a combination of polypeptide-encoding nucleic acid sequences and non-coding nucleic acid sequences.

A vector can contain between 1 and 30 payload-encoding nucleic acid sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different payload-encoding nucleic acid sequences, 6, 7, 8, 9, 10 11, 12, 13, or 14 different payload-encoding nucleic acid sequences, or 12, 13 or 14 different payload-encoding nucleic acid sequences. Payload-encoding nucleic acid sequences can refer to the payload encoding portion of a “cassette.” Features of a cassette are described in greater detail herein. A cassette can contain two or more payload-encoding nucleic acid sequences linked together in a cassette (e.g., as an illustrative non-limiting example, concatenated antigen-encoding nucleic acid sequence encoding concatenated T cell epitopes)

A vector can contain between 1 and 30 distinct payload-encoding nucleic acid sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more distinct payload-encoding nucleic acid sequences, 6, 7, 8, 9, 10 11, 12, 13, or 14 distinct payload-encoding nucleic acid sequences, or 12, 13 or 14 distinct payload-encoding nucleic acid sequences. Payload-encoding nucleic acid sequences can refer to sequences for individual payload sequences, e.g., as an illustrative non-limiting example, each of the concatenated T cell epitopes of two or more payload-encoding nucleic acid sequences linked together in a cassette.

Antigens can include nucleotides or polypeptides. For example, an antigen can be an RNA sequence that encodes for a polypeptide sequence. Antigens useful in vaccines can therefore include nucleotide sequences or polypeptide sequences. Antigens that can be used for cancer vaccines are described in international patent application publication WO/2019/226941, which is herein incorporated by reference, in its entirety, for all purposes.

Disclosed herein are isolated peptides that comprise tumor specific mutations identified by the methods disclosed herein, peptides that comprise known tumor specific mutations, and mutant polypeptides or fragments thereof identified by methods disclosed herein. Neoantigen peptides can be described in the context of their coding sequence where a neoantigen includes the nucleotide sequence (e.g., DNA or RNA) that codes for the related polypeptide sequence.

Also disclosed herein are peptides derived from any polypeptide known to or have been found to have altered expression in a tumor cell or cancerous tissue in comparison to a normal cell or tissue, for example any polypeptide known to or have been found to be aberrantly expressed in a tumor cell or cancerous tissue in comparison to a normal cell or tissue. Suitable polypeptides from which the antigenic peptides can be derived can be found for example in the COSMIC database. COSMIC curates comprehensive information on somatic mutations in human cancer. The peptide contains the tumor specific mutation. Tumor antigens (e.g., shared tumor antigens and tumor neoantigens) can include, but are not limited to, those described in U.S. application Ser. No. 17/058,128, herein incorporated by reference for all purposes. Antigen peptides can be described in the context of their coding sequence where an antigen includes the nucleotide sequence (e.g., DNA or RNA) that codes for the related polypeptide sequence.

One or more polypeptides encoded by an antigen nucleotide sequence can comprise at least one of: a binding affinity with MHC with an IC50 value of less than 1000 nM, for MHC Class I peptides a length of 8-15, 8, 9, 10, 11, 12, 13, 14, or 15 amino acids, presence of sequence motifs within or near the peptide promoting proteasome cleavage, and presence or sequence motifs promoting TAP transport. For MHC Class II peptides a length 6-30, 6, 7, 8, 9, 10, 11, 12, 13, 14, 15, 16, 17, 18, 19, 20, 21, 22, 23, 24, 25, 26, 27, 28, 29, or 30 amino acids, presence of sequence motifs within or near the peptide promoting cleavage by extracellular or lysosomal proteases (e.g., cathepsins) or HLA-DM catalyzed HLA binding.

One or more antigens can be presented on the surface of a tumor.

One or more antigens can be is immunogenic in a subject having a tumor, e.g., capable of eliciting a T cell response or a B cell response in the subject.

One or more antigens that induce an autoimmune response in a subject can be excluded from consideration in the context of vaccine generation for a subject having a tumor.

The size of at least one antigenic peptide molecule can comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120 or greater amino molecule residues, and any range derivable therein. In specific embodiments the antigenic peptide molecules are equal to or less than 50 amino acids.

Antigenic peptides and polypeptides can be for MHC Class I 15 residues or less in length and usually consist of between about 8 and about 11 residues, particularly 9 or 10 residues; for MHC Class II, 6-30 residues, inclusive.

Antigenic peptides and polypeptides can be presented on an HLA protein. In some aspects, antigenic peptides and polypeptides are presented on an HLA protein with greater affinity than a wild-type peptide. In some aspects, an antigenic peptide or polypeptide can have an IC50 of at least less than 5000 nM, at least less than 1000 nM, at least less than 500 nM, at least less than 250 nM, at least less than 200 nM, at least less than 150 nM, at least less than 100 nM, at least less than 50 nM or less.

In some aspects, antigenic peptides and polypeptides do not induce an autoimmune response and/or invoke immunological tolerance when administered to a subject.

Also provided are compositions comprising at least two or more antigenic peptides. In some embodiments the composition contains at least two distinct peptides. At least two distinct peptides can be derived from the same polypeptide. By distinct polypeptides is meant that the peptide varies by length, amino acid sequence, or both. The peptides are derived from any polypeptide known to or have been found to contain a tumor specific mutation or peptides derived from any polypeptide known to or have been found to have altered expression in a tumor cell or cancerous tissue in comparison to a normal cell or tissue, for example any polypeptide known to or have been found to be aberrantly expressed in a tumor cell or cancerous tissue in comparison to a normal cell or tissue. Suitable polypeptides from which the antigenic peptides can be derived can be found for example in the COSMIC database or the AACR Genomics Evidence Neoplasia Information Exchange (GENIE) database. COSMIC curates comprehensive information on somatic mutations in human cancer. AACR GENIE aggregates and links clinical-grade cancer genomic data with clinical outcomes from tens of thousands of cancer patients. The peptide contains the tumor specific mutation. In some aspects, the tumor specific mutation is a driver mutation for a particular cancer type.

Also disclosed herein are peptides derived from any polypeptide associated with an infectious disease organism, an infection in a subject, or an infected cell of a subject. Antigens can be derived from nucleotide sequences or polypeptide sequences of an infectious disease organism. Polypeptide sequences of an infectious disease organism include, but are not limited to, a pathogen-derived peptide, a virus-derived peptide, a bacteria-derived peptide, a fungus-derived peptide, and/or a parasite-derived peptide. Infectious disease organism include, but are not limited to, Severe acute respiratory syndrome-related coronavirus (SARS), severe acute respiratory syndrome coronavirus 2 (SARS-CoV-2), Ebola, HIV, Hepatitis B virus (HBV), influenza, Hepatitis C virus (HCV), Human papillomavirus (HPV), Cytomegalovirus (CMV), Chikungunya virus, Respiratory syncytial virus (RSV), Dengue virus, an orthymyxoviridae family virus, and tuberculosis.

Disclosed herein are isolated peptides that comprise infectious disease organism specific antigens or epitopes identified by the methods disclosed herein, peptides that comprise known infectious disease organism specific antigens or epitopes, and mutant polypeptides or fragments thereof identified by methods disclosed herein. Antigen peptides can be described in the context of their coding sequence where an antigen includes the nucleotide sequence (e.g., DNA or RNA) that codes for the related polypeptide sequence.

Vectors and associated compositions described herein can be used to deliver antigens from any organism, including their toxins or other by-products, to prevent and/or treat infection or other adverse reactions associated with the organism or its by-product.

Antigens that can be incorporated into a vaccine (e.g., encoded in a cassette) include immunogens which are useful to immunize a human or non-human animal against viruses, such as pathogenic viruses which infect human and non-human vertebrates. Antigens may be selected from a variety of viral families. Example of desirable viral families against which an immune response would be desirable include, the picornavirus family, which includes the genera rhinoviruses, which are responsible for about 50% of cases of the common cold; the genera enteroviruses, which include polioviruses, coxsackieviruses, echoviruses, and human enteroviruses such as hepatitis A virus; and the genera apthoviruses, which are responsible for foot and mouth diseases, primarily in non-human animals. Within the picornavirus family of viruses, target antigens include the VP1, VP2, VP3, VP4, and VPG. Another viral family includes the calcivirus family, which encompasses the Norwalk group of viruses, which are an important causative agent of epidemic gastroenteritis. Still another viral family desirable for use in targeting antigens for stimulating immune responses in humans and non-human animals is the togavirus family, which includes the genera alphavirus, which include Sindbis viruses, RossRiver virus, and Venezuelan, Eastern & Western Equine encephalitis, and rubivirus, including Rubella virus. The Flaviviridae family includes dengue, yellow fever, Japanese encephalitis, St. Louis encephalitis and tick borne encephalitis viruses. Other target antigens may be generated from the Hepatitis C or the coronavirus family, which includes a number of non-human viruses such as infectious bronchitis virus (poultry), porcine transmissible gastroenteric virus (pig), porcine hemagglutinating encephalomyelitis virus (pig), feline infectious peritonitis virus (cats), feline enteric coronavirus (cat), canine coronavirus (dog), and human respiratory coronaviruses, which may cause the common cold and/or non-A, B or C hepatitis. Within the coronavirus family, target antigens include the E1 (also called M or matrix protein), E2 (also called S or Spike protein), E3 (also called HE or hemagglutin-elterose) glycoprotein (not present in all coronaviruses), or N (nucleocapsid). Still other antigens may be targeted against the rhabdovirus family, which includes the genera vesiculovirus (e.g., Vesicular Stomatitis Virus), and the general lyssavirus (e.g., rabies). Within the rhabdovirus family, suitable antigens may be derived from the G protein or the N protein. The family filoviridae, which includes hemorrhagic fever viruses such as Marburg and Ebola virus, may be a suitable source of antigens. The paramyxovirus family includes parainfluenza Virus Type 1, parainfluenza Virus Type 3, bovine parainfluenza Virus Type 3, rubulavirus (mumps virus), parainfluenza Virus Type 2, parainfluenza virus Type 4, Newcastle disease virus (chickens), rinderpest, morbillivirus, which includes measles and canine distemper, and pneumovirus, which includes respiratory syncytial virus (e.g., the glyco-(G) protein and the fusion (F) protein, for which sequences are available from GenBank). Influenza virus is classified within the family orthomyxovirus and can be suitable source of antigens (e.g., the HA protein, the N1 protein). The bunyavirus family includes the genera bunyavirus (California encephalitis, La Crosse), phlebovirus (Rift Valley Fever), hantavirus (puremala is a hemahagin fever virus), nairovirus (Nairobi sheep disease) and various unassigned bungaviruses. The arenavirus family provides a source of antigens against LCM and Lassa fever virus. The reovirus family includes the genera reovirus, rotavirus (which causes acute gastroenteritis in children), orbiviruses, and cultivirus (Colorado Tick fever, Lebombo (humans), equine encephalosis, blue tongue). The retrovirus family includes the sub-family oncorivirinal which encompasses such human and veterinary diseases as feline leukemia virus, HTLVI and HTLVII, lentivirinal (which includes human immunodeficiency virus (HIV), simian immunodeficiency virus (SIV), feline immunodeficiency virus (FIV), equine infectious anemia virus, and spumavirinal). Among the lentiviruses, many suitable antigens have been described and can readily be selected. Examples of suitable HIV and SIV antigens include, without limitation the gag, pol, Vif, Vpx, VPR, Env, Tat, Nef, and Rev proteins, as well as various fragments thereof. For example, suitable fragments of the Env protein may include any of its subunits such as the gp120, gp160, gp41, or smaller fragments thereof, e.g., of at least about 8 amino acids in length. Similarly, fragments of the tat protein may be selected. [See, U.S. Pat. Nos. 5,891,994 and 6,193,981.] See, also, the HIV and SIV proteins described in D. H. Barouch et al, J. Virol., 75(5):2462-2467 (March 2001), and R. R. Amara, et al, Science, 292:69-74 (6 Apr. 2001). In another example, the HIV and/or SIV immunogenic proteins or peptides may be used to form fusion proteins or other immunogenic molecules. See, e.g., the HIV-1 Tat and/or Nef fusion proteins and immunization regimens described in WO 01/54719, published Aug. 2, 2001, and WO 99/16884, published Apr. 8, 1999. The invention is not limited to the HIV and/or SIV immunogenic proteins or peptides described herein. In addition, a variety of modifications to these proteins have been described or could readily be made by one of skill in the art. See, e.g., the modified gag protein that is described in U.S. Pat. No. 5,972,596. Further, any desired HIV and/or SIV immunogens may be delivered alone or in combination. Such combinations may include expression from a single vector or from multiple vectors. The papovavirus family includes the sub-family polyomaviruses (BKU and JCU viruses) and the sub-family papillomavirus (associated with cancers or malignant progression of papilloma). The adenovirus family includes viruses (EX, AD7, ARD, O.B.) which cause respiratory disease and/or enteritis. The parvovirus family feline parvovirus (feline enteritis), feline panleucopeniavirus, canine parvovirus, and porcine parvovirus. The herpesvirus family includes the sub-family alphaherpesvirinae, which encompasses the genera simplexvirus (HSVI, HSVII), varicellovirus (pseudorabies, varicella zoster) and the sub-family betaherpesvirinae, which includes the genera cytomegalovirus (Human CMV), muromegalovirus) and the sub-family gammaherpesvirinae, which includes the genera lymphocryptovirus, EBV (Burkitts lymphoma), infectious rhinotracheitis, Marek's disease virus, and rhadinovirus. The poxvirus family includes the sub-family chordopoxyirinae, which encompasses the genera orthopoxvirus (Variola (Smallpox) and Vaccinia (Cowpox)), parapoxvirus, avipoxvirus, capripoxvirus, leporipoxvirus, suipoxvirus, and the sub-family entomopoxyirinae. The hepadnavirus family includes the Hepatitis B virus. One unclassified virus which may be suitable source of antigens is the Hepatitis delta virus. Still other viral sources may include avian infectious bursal disease virus and porcine respiratory and reproductive syndrome virus. The alphavirus family includes equine arteritis virus and various Encephalitis viruses.

Antigens that can be incorporated into a vaccine (e.g., encoded in a cassette) also include immunogens which are useful to immunize a human or non-human animal against pathogens including bacteria, fungi, parasitic microorganisms or multicellular parasites which infect human and non-human vertebrates. Examples of bacterial pathogens include pathogenic gram-positive cocci include pneumococci; staphylococci; and streptococci. Pathogenic gram-negative cocci include meningococcus; gonococcus. Pathogenic enteric gram-negative bacilli include enterobacteriaceae; pseudomonas, acinetobacteria and eikenella; melioidosis; salmonella; shigella; haemophilus (Haemophilus influenzae, Haemophilus somnus); moraxella; H. ducreyi (which causes chancroid); brucella; Franisella tularensis (which causes tularemia); yersinia (pasteurella); streptobacillus moniliformis and spirillum. Gram-positive bacilli include Listeria monocytogenes; erysipelothrix rhusiopathiae; Corynebacterium diphtheria (diphtheria); cholera; B. anthracis (anthrax); donovanosis (granuloma inguinale); and bartonellosis. Diseases caused by pathogenic anaerobic bacteria include tetanus; botulism; other clostridia; tuberculosis; leprosy; and other mycobacteria. Examples of specific bacterium species are, without limitation, Streptococcus pneumoniae, Streptococcus pyogenes, Streptococcus agalactiae, Streptococcus faecalis, Moraxella catarrhalis, Helicobacter pylori, Neisseria meningitidis, Neisseria gonorrhoeae, Chlamydia trachomatis, Chlamydia pneumoniae, Chlamydia psittaci, Bordetella pertussis, Salmonella typhi, Salmonella typhimurium, Salmonella choleraesuis, Escherichia coli, Shigella, Vibrio cholerae, Corynebacterium diphtheriae, Mycobacterium tuberculosis, Mycobacterium avium, Mycobacterium intracellulare complex, Proteus mirabilis, Proteus vulgaris, Staphylococcus aureus, Clostridium tetani, Leptospira interrogans, Borrelia burgdorferi, Pasteurella haemolytica, Pasteurella multocida, Actinobacillus pleuropneumoniae and Mycoplasma gallisepticum. Pathogenic spirochetal diseases include syphilis; treponematoses: yaws, pinta and endemic syphilis; and leptospirosis. Other infections caused by higher pathogen bacteria and pathogenic fungi include actinomycosis; nocardiosis; cryptococco sis (Cryptococcus), blastomycosis (Blastomyces), histoplasmosis (Histoplasma) and coccidioidomycosis (Coccidiodes); candidiasis (Candida), aspergillosis (Aspergillis), and mucormycosis; sporotrichosis; paracoccidiodomycosis, petriellidiosis, torulopsosis, mycetoma and chromomycosis; and dermatophytosis. Rickettsial infections include Typhus fever, Rocky Mountain spotted fever, Q fever, and Rickettsialpox. Examples of mycoplasma and chlamydial infections include Mycoplasma pneumoniae; lymphogranuloma venereum; psittacosis; and perinatal chlamydial infections. Pathogenic eukaryotes encompass pathogenic protozoans and helminths and infections produced thereby include: amebiasis; malaria; leishmaniasis (e.g., caused by Leishmania major); trypanosomiasis; toxoplasmosis (e.g., caused by Toxoplasma gondii); Pneumocystis carinii; Trichans; Toxoplasma gondii; babesiosis; giardiasis (e.g., caused by Giardia); trichinosis (e.g., caused by Trichomonas); filariasis; schistosomiasis (e.g., caused by Schistosoma); nematodes; trematodes or flukes; and cestode (tapeworm) infections. Other parasitic infections may be caused by Ascaris, Trichuris, Cryptosporidium, and Pneumocystis carinii, among others.

Also disclosed herein are peptides derived from any polypeptide associated with an infectious disease organism, an infection in a subject, or an infected cell of a subject. Antigens can be derived from nucleic acid sequences or polypeptide sequences of an infectious disease organism. Polypeptide sequences of an infectious disease organism include, but are not limited to, a pathogen-derived peptide, a virus-derived peptide, a bacteria-derived peptide, a fungus-derived peptide, and/or a parasite-derived peptide. Infectious disease organism include, but are not limited to, Severe acute respiratory syndrome-related coronavirus (SARS), severe acute respiratory syndrome coronavirus 2 (SARS-CoV-2), Ebola, HIV, Hepatitis B virus (HBV), influenza, Hepatitis C virus (HCV), Human papillomavirus (HPV), Cytomegalovirus (CMV), Chikungunya virus, Respiratory syncytial virus (RSV), Dengue virus, an orthymyxoviridae family virus, and tuberculosis.

Antigens can be selected that are predicted to be presented on the cell surface of a cell, such as a tumor cell, an infected cell, or an immune cell, including professional antigen presenting cells such as dendritic cells. Antigens can be selected that are predicted to be immunogenic.

One or more polypeptides encoded by an antigen nucleotide sequence can comprise at least one of: a binding affinity with MHC with an IC₅₀ value of less than 1000 nM, for MHC Class I peptides a length of 8-15, 8, 9, 10, 11, 12, 13, 14, or 15 amino acids, presence of sequence motifs within or near the peptide promoting proteasome cleavage, and presence or sequence motifs promoting TAP transport. For MHC Class II peptides a length 6-30, 6, 7, 8, 9, 10, 11, 12, 13, 14, 15, 16, 17, 18, 19, 20, 21, 22, 23, 24, 25, 26, 27, 28, 29, or 30 amino acids, presence of sequence motifs within or near the peptide promoting cleavage by extracellular or lysosomal proteases (e.g., cathepsins) or HLA-DM catalyzed HLA binding.

One or more antigens can be presented on the surface of a tumor. One or more antigens can be presented on the surface of an infected cell.

One or more antigens can be immunogenic in a subject having a tumor, e.g., capable of stimulating a T cell response and/or a B cell response in the subject. One or more antigens can be immunogenic in a subject having or suspected to have an infection, e.g., capable of stimulating a T cell response and/or a B cell response in the subject. One or more antigens can be immunogenic in a subject at risk of an infection, e.g., capable of stimulating a T cell response and/or a B cell response in the subject that provides immunological protection (i.e., immunity) against the infection, e.g., such as stimulating the production of memory T cells, memory B cells, or antibodies specific to the infection.

One or more antigens can be capable of stimulating a B cell response, such as the production of antibodies that recognize the one or more antigens (e.g., antibodies that recognize a tumor or an infectious disease antigen). Antibodies can recognize linear polypeptide sequences or recognize secondary and tertiary structures. Accordingly, B cell antigens can include linear polypeptide sequences or polypeptides having secondary and tertiary structures, including, but not limited to, full-length proteins, protein subunits, protein domains, or any polypeptide sequence known or predicted to have secondary and tertiary structures. Antigens capable of stimulating a B cell response to a tumor or an infectious disease antigen can be an antigen found on the surface of tumor cell or an infectious disease organism, respectively. Antigens capable of eliciting a B cell response to a tumor or an infectious disease antigen can be an intracellular neoantigen expressed in a tumor or an infectious disease organism, respectively.

One or more antigens can include a combination of antigens capable of stimulating a T cell response (e.g., peptides including predicted T cell epitope sequences) and distinct antigens capable of stimulating a B cell response (e.g., full-length proteins, protein subunits, protein domains).

One or more antigens that stimulate an autoimmune response in a subject can be excluded from consideration in the context of vaccine generation for a subject.

The size of at least one antigenic peptide molecule (e.g., an epitope sequence) can comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120 or greater amino molecule residues, and any range derivable therein. In specific embodiments the antigenic peptide molecules are equal to or less than 50 amino acids.

Antigenic peptides and polypeptides can be for MHC Class I 15 residues or less in length and usually include between about 8 and about 11 residues, particularly 9 or 10 residues; for MHC Class II, 6-30 residues, inclusive.

If desirable, a longer peptide can be designed in several ways. In one case, when presentation likelihoods of peptides on HLA alleles are predicted or known, a longer peptide could include either: (1) individual presented peptides with an extensions of 2-5 amino acids toward the N- and C-terminus of each corresponding gene product; (2) a concatenation of some or all of the presented peptides with extended sequences for each. In another case, when sequencing reveals a long (>10 residues) neoepitope sequence present in the tumor (e.g. due to a frameshift, read-through or intron inclusion that leads to a novel peptide sequence), a longer peptide would include: (3) the entire stretch of novel tumor-specific or infectious disease-specific amino acids—thus bypassing the need for computational or in vitro test-based selection of the strongest HLA-presented shorter peptide. In both cases, use of a longer peptide allows endogenous processing by patient cells and may lead to more effective antigen presentation and stimulation of T cell responses. Longer peptides can also include a full-length protein, a protein subunit, a protein domain, and combinations thereof of a peptide, such as those expressed in a tumor or an infectious disease organism, respectively. Longer peptides (e.g., full-length protein, protein subunit, or protein domain) and combinations thereof can be included to stimulate a B cell response.

Antigenic peptides and polypeptides can be presented on an HLA protein. In some aspects, antigenic peptides and polypeptides are presented on an HLA protein with greater affinity than a wild-type peptide. In some aspects, an antigenic peptide or polypeptide can have an IC50 of at least less than 5000 nM, at least less than 1000 nM, at least less than 500 nM, at least less than 250 nM, at least less than 200 nM, at least less than 150 nM, at least less than 100 nM, at least less than 50 nM or less.

In some aspects, antigenic peptides and polypeptides do not induce an autoimmune response and/or invoke immunological tolerance when administered to a subject.

Also provided are compositions comprising at least two or more antigenic peptides. In some embodiments the composition contains at least two distinct peptides. At least two distinct peptides can be derived from the same polypeptide. By distinct polypeptides is meant that the peptide varies by length, amino acid sequence, or both. A peptide can include a tumor-specific mutation. Tumor-specific peptides can be derived from any polypeptide known to or have been found to contain a tumor specific mutation or peptides derived from any polypeptide known to or have been found to have altered expression in a tumor cell or cancerous tissue in comparison to a normal cell or tissue, for example any polypeptide known to or have been found to be aberrantly expressed in a tumor cell or cancerous tissue in comparison to a normal cell or tissue. The peptides can be derived from any polypeptide known to or suspected to be associated with an infectious disease organism, or peptides derived from any polypeptide known to or have been found to have altered expression in an infected cell in comparison to a normal cell or tissue (e.g., an infectious disease polynucleotide or polypeptide, including infectious disease polynucleotides or polypeptides with expression restricted to a host cell). Suitable polypeptides from which the antigenic peptides can be derived can be found for example in the COSMIC database or the AACR Genomics Evidence Neoplasia Information Exchange (GENIE) database. COSMIC curates comprehensive information on somatic mutations in human cancer. AACR GENIE aggregates and links clinical-grade cancer genomic data with clinical outcomes from tens of thousands of cancer patients. In some aspects, the tumor specific mutation is a driver mutation for a particular cancer type.

Antigenic peptides and polypeptides having a desired activity or property can be modified to provide certain desired attributes, e.g., improved pharmacological characteristics, while increasing or at least retaining substantially all of the biological activity of the unmodified peptide to bind the desired MHC molecule and activate the appropriate T cell. For instance, antigenic peptide and polypeptides can be subject to various changes, such as substitutions, either conservative or non-conservative, where such changes might provide for certain advantages in their use, such as improved MHC binding, stability or presentation. By conservative substitutions is meant replacing an amino acid residue with another which is biologically and/or chemically similar, e.g., one hydrophobic residue for another, or one polar residue for another. The substitutions include combinations such as Gly, Ala; Val, Ile, Leu, Met; Asp, Glu; Asn, Gln; Ser, Thr; Lys, Arg; and Phe, Tyr. The effect of single amino acid substitutions may also be probed using D-amino acids. Such modifications can be made using well known peptide synthesis procedures, as described in e.g., Merrifield, Science 232:341-347 (1986), Barany & Merrifield, The Peptides, Gross & Meienhofer, eds. (N.Y., Academic Press), pp. 1-284 (1979); and Stewart & Young, Solid Phase Peptide Synthesis, (Rockford, Ill., Pierce), 2d Ed. (1984).

Modifications of peptides and polypeptides with various amino acid mimetics or unnatural amino acids can be particularly useful in increasing the stability of the peptide and polypeptide in vivo. Stability can be assayed in a number of ways. For instance, peptidases and various biological media, such as human plasma and serum, have been used to test stability. See, e.g., Verhoef et al., Eur. J. Drug Metab Pharmacokin. 11:291-302 (1986). Half-life of the peptides can be conveniently determined using a 25% human serum (v/v) assay. The protocol is generally as follows. Pooled human serum (Type AB, non-heat inactivated) is delipidated by centrifugation before use. The serum is then diluted to 25% with RPMI tissue culture media and used to test peptide stability. At predetermined time intervals a small amount of reaction solution is removed and added to either 6% aqueous trichloroacetic acid or ethanol. The cloudy reaction sample is cooled (4 degrees C.) for 15 minutes and then spun to pellet the precipitated serum proteins. The presence of the peptides is then determined by reversed-phase HPLC using stability-specific chromatography conditions.

The peptides and polypeptides can be modified to provide desired attributes other than improved serum half-life. For instance, the ability of the peptides to stimulate CTL activity can be enhanced by linkage to a sequence which contains at least one epitope that is capable of stimulating a T helper cell response. Immunogenic peptides/T helper conjugates can be linked by a spacer molecule. The spacer is typically comprised of relatively small, neutral molecules, such as amino acids or amino acid mimetics, which are substantially uncharged under physiological conditions. The spacers are typically selected from, e.g., Ala, Gly, or other neutral spacers of nonpolar amino acids or neutral polar amino acids. It will be understood that the optionally present spacer need not be comprised of the same residues and thus can be a hetero- or homo-oligomer. When present, the spacer will usually be at least one or two residues, more usually three to six residues. Alternatively, the peptide can be linked to the T helper peptide without a spacer.

An antigenic peptide can be linked to the T helper peptide either directly or via a spacer either at the amino or carboxy terminus of the peptide. The amino terminus of either the antigenic peptide or the T helper peptide can be acylated. Exemplary T helper peptides include tetanus toxoid 830-843, influenza 307-319, malaria circumsporozoite 382-398 and 378-389.

Proteins or peptides can be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and can be found at computerized databases known to those of ordinary skill in the art. One such database is the National Center for Biotechnology Information's Genbank and GenPept databases located at the National Institutes of Health website. The coding regions for known genes can be amplified and/or expressed using the techniques disclosed herein or as would be known to those of ordinary skill in the art. Alternatively, various commercial preparations of proteins, polypeptides and peptides are known to those of skill in the art.

In a further aspect an antigen includes a nucleic acid (e.g., polynucleotide) that encodes an antigenic peptide or portion thereof. The polynucleotide can be, e.g., DNA, cDNA, PNA, CNA, RNA (e.g., mRNA), either single- and/or double-stranded, or native or stabilized forms of polynucleotides, such as, e.g., polynucleotides with a phosphorothioate backbone, or combinations thereof and it may or may not contain introns. A polynucleotide sequence encoding an antigen can be sequence-optimized to improve expression, such as through improving transcription, translation, post-transcriptional processing, and/or RNA stability. For example, polynucleotide sequence encoding an antigen can be codon-optimized. “Codon-optimization” herein refers to replacing infrequently used codons, with respect to codon bias of a given organism, with frequently used synonymous codons. Polynucleotide sequences can be optimized to improve post-transcriptional processing, for example optimized to reduce unintended splicing, such as through removal of splicing motifs (e.g., canonical and/or cryptic/non-canonical splice donor, branch, and/or acceptor sequences) and/or introduction of exogenous splicing motifs (e.g., splice donor, branch, and/or acceptor sequences) to bias favored splicing events. Exogenous intron sequences include, but are not limited to, those derived from SV40 (e.g., an SV40 mini-intron) and derived from immunoglobulins (e.g., human β-globin gene). Exogenous intron sequences can be incorporated between a promoter/enhancer sequence and the antigen(s) sequence. Exogenous intron sequences for use in expression vectors are described in more detail in Callendret et al. (Virology. 2007 Jul. 5; 363(2): 288-302), herein incorporated by reference for all purposes. Polynucleotide sequences can be optimized to improve transcript stability, for example through removal of RNA instability motifs (e.g., AU-rich elements and 3′ UTR motifs) and/or repetitive nucleotide sequences. Polynucleotide sequences can be optimized to improve accurate transcription, for example through removal of cryptic transcriptional initiators and/or terminators. Polynucleotide sequences can be optimized to improve translation and translational accuracy, for example through removal of cryptic AUG start codons, premature polyA sequences, and/or secondary structure motifs. Polynucleotide sequences can be optimized to improve nuclear export of transcripts, such as through addition of a Constitutive Transport Element (CTE), RNA Transport Element (RTE), or Woodchuck Posttranscriptional Regulatory Element (WPRE). Nuclear export signals for use in expression vectors are described in more detail in Callendret et al. (Virology. 2007 Jul. 5; 363(2): 288-302), herein incorporated by reference for all purposes. Polynucleotide sequences can be optimized with respect to GC content, for example to reflect the average GC content of a given organism. Sequence optimization can balance one or more sequence properties, such as transcription, translation, post-transcriptional processing, and/or RNA stability. Sequence optimization can generate an optimal sequence balancing each of transcription, translation, post-transcriptional processing, and RNA stability. Sequence optimization algorithms are known to those of skill in the art, such as GeneArt (Thermo Fisher), Codon Optimization Tool (IDT), Cool Tool (University of Singapore), SGI-DNA (La Jolla California). One or more regions of an antigen-encoding protein can be sequence-optimized separately.

A still further aspect provides an expression vector capable of expressing a polypeptide or portion thereof. Expression vectors for different cell types are well known in the art and can be selected without undue experimentation. Generally, DNA is inserted into an expression vector, such as a plasmid, in proper orientation and correct reading frame for expression. If necessary, DNA can be linked to the appropriate transcriptional and translational regulatory control nucleotide sequences recognized by the desired host, although such controls are generally available in the expression vector. The vector is then introduced into the host through standard techniques. Guidance can be found e.g. in Sambrook et al. (1989) Molecular Cloning, A Laboratory Manual, Cold Spring Harbor Laboratory, Cold Spring Harbor, N.Y.

### Cassette

The methods employed for the selection of one or more payloads, the cloning and construction of a “cassette” and its insertion into a viral vector are within the skill in the art given the teachings provided herein. By “payload cassette” or “cassette” or “antigen cassette” is meant the combination of a selected payload or plurality of payloads (e.g., payload-encoding nucleic acid sequences, such as antigen-encoding nucleic acid sequences) and the other regulatory elements necessary to transcribe the payload(s) and express the transcribed product. The selected payload or plurality of payloads can refer to distinct payload sequences, e.g., a payload-encoding nucleic acid sequence in the cassette can encode a payload-encoding nucleic acid sequence (or plurality of payload-encoding nucleic acid sequences) such that the payloads are transcribed and expressed. A payload or plurality of payloads can be operatively linked to regulatory components in a manner which permits transcription. Such components include conventional regulatory elements that can drive expression of the payload(s) in a cell transfected with the viral vector. Thus, the payload cassette can also contain a selected promoter which is linked to the payload(s) and located, with other, optional regulatory elements, within the selected viral sequences of the recombinant vector. A cassette can include one or more payloads, such as one or more sequences encoding any of the payloads described herein. A cassette can have one or more payload-encoding nucleic acid sequences, such as a cassette containing multiple payload-encoding nucleic acid sequences each independently operably linked to separate promoters and/or linked together using other multicistronic systems, such as 2A ribosome skipping sequence elements (e.g., E2A, P2A, F2A, or T2A sequences) or Internal Ribosome Entry Site (IRES) sequence elements. A linker can also have a cleavage site, such as a TEV or furin cleavage site. Linkers with cleavage sites can be used in combination with other elements, such as those in a multicistronic system. In a non-limiting illustrative example, a furin protease cleavage site can be used in conjunction with a 2A ribosome skipping sequence element such that the furin protease cleavage site is configured to facilitate removal of the 2A sequence following translation.

In a cassette containing more than one payload-encoding nucleic acid sequences, each payload-encoding nucleic acid sequence can be concatenated (e.g., in an illustrative non-limiting example, concatenated payload-encoding nucleic acid sequences encoding concatenated T cell epitopes). In illustrative examples of multicistronic formats, cassettes encoding payloads are configured as follows: (1) endogenous 26S promoter-payload 1-T2A-payload 2 protein, or (2) endogenous 26S promoter-payload 1-26S promoter-payload 2. In further illustrative examples of multicistronic formats, cassettes encoding SARS-CoV-2 payloads are configured as follows: (1) endogenous 26S promoter-Spike protein-T2A-Membrane protein, or (2) endogenous 26S promoter-Spike protein-26S promoter-concatenated T cell epitopes.

In addition to the subgenomic alphavirus-derived promoter described herein, additional promoter or promoter elements can be employed. Useful promoters can be constitutive promoters or regulated (inducible) promoters, which will enable control of the amount of payload(s) to be expressed. For example, a desirable promoter is that of the cytomegalovirus immediate early promoter/enhancer [see, e.g., Boshart et al, Cell, 41:521-530 (1985)]. Another desirable promoter includes the Rous sarcoma virus LTR promoter/enhancer. Still another promoter/enhancer sequence is the chicken cytoplasmic beta-actin promoter [T. A. Kost et al, Nucl. Acids Res., 11(23):8287 (1983)]. Other suitable or desirable promoters can be selected by one of skill in the art.

A cassette can also include nucleic acid sequences heterologous to the viral vector sequences including sequences providing signals for efficient polyadenylation of the transcript (poly(A), poly-A or pA) and introns with functional splice donor and acceptor sites. A common poly-A sequence which is employed in the exemplary vectors of this invention is that derived from the papovavirus SV-40. A poly-A sequence (e.g., a non-native poly-A) generally can be inserted in the cassette following the payload-based sequences and before the viral vector sequences. A common intron sequence can also be derived from SV-40 and is referred to as the SV-40 T intron sequence. A cassette can also contain such an intron, located between the promoter/enhancer sequence and the payload(s). Selection of these and other common vector elements are conventional [see, e.g., Sambrook et al, “Molecular Cloning. A Laboratory Manual.”, 2d edit., Cold Spring Harbor Laboratory, New York (1989) and references cited therein] and many such sequences are available from commercial and industrial sources as well as from Genbank.

A cassette can have one or more payloads (e.g., one or more payload-encoding nucleic acid sequences). For example, a given cassette can include 1-10, 1-20, 1-30, 10-20, 15-25, 15-20, 1, 2, 3, 4, 5, 6, 7, 8, 9, 10, 11, 12, 13, 14, 15, 16, 17, 18, 19, 20, or more payloads. Payloads can be linked directly to one another. Payloads can also be linked to one another with linkers. Payloads can be in any orientation relative to one another including N to C or C to N.

As described elsewhere herein, a cassette can be located in the site of any selected deletion in a viral vector, such as the deleted structural proteins of a VEE backbone or the site of the E1 gene region deletion or E3 gene region deletion of a ChAd-based vector, among others which may be selected.

The multicistronic samRNA vectors can be described using the following formula to describe the ordered sequence of each element, from 5′ to 3′:

P₁-(L5_{b}-N_{c}-L3_{d})_{X}-P₂-(L5_{b}-N_{c}-L3_{d})_{X}-Pₐ-(L5_{b}-N_{c}-L3_{d})_{X}-(G5ₑ-U_{f})Y-G3_{g}
wherein P₁ comprises the SGP1 subgenomic promoter, P₂ comprises the SGP2 subgenomic promoter where for Pₐ a=0 or 1 for additional cassettes, N comprises a payload-encoding nucleic acid sequences, where c=1, L5 comprises the 5′ linker sequence, where b=0 or 1, L3 comprises the 3′ linker sequence, where d=0 or 1, G5 comprises one of the at least one nucleic acid sequences encoding a GPGPG amino acid linker (SEQ ID NO: 1), where e=0 or 1, G3 comprises one of the at least one nucleic acid sequences encoding a GPGPG amino acid linker (SEQ ID NO: 1), where g=0 or 1, U comprises one of the at least one MHC class II epitope-encoding nucleic acid sequence, where f=1, X=1 to 400, where for each X the corresponding N_{c} is a corresponding payload-encoding nucleic acid sequence, and Y=0, 1, or 2, where for each Y the corresponding U_{f} is a universal MHC class II epitope-encoding nucleic acid sequence, optionally wherein the at least one universal sequence comprises at least one of Tetanus toxoid and PADRE.

A payload encoding sequence (e.g., cassette or one or more of the nucleic acid sequences encoding a payload in the cassette) can be described using the following formula to describe the ordered sequence of each element, from 5′ to 3′:

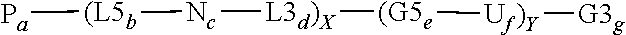

wherein P comprises the second promoter nucleotide sequence, where a=0 or 1, where c=1, N comprises one of the payload-derived nucleic acid sequences described herein (e.g., any of the antigen-encoding nucleic acid sequences described herein), L5 comprises the 5′ linker sequence, where b=0 or 1, L3 comprises the 3′ linker sequence, where d=0 or 1, G5 comprises one of the at least one nucleic acid sequences encoding a GPGPG amino acid linker (SEQ ID NO: 1), where e=0 or 1, G3 comprises one of the at least one nucleic acid sequences encoding a GPGPG amino acid linker (SEQ ID NO: 1), where g=0 or 1, U comprises one of the at least one MHC class II epitope-encoding nucleic acid sequence, where f=1, X=1 to 400, where for each X the corresponding N_{c} is a payload-encoding nucleic acid sequence, and Y=0, 1, or 2, where for each Y the corresponding U_{f} is a (1) universal MHC class II epitope-encoding nucleic acid sequence, optionally wherein the at least one universal sequence comprises at least one of Tetanus toxoid and PADRE, or (2) a MHC class II epitope-encoding nucleic acid sequence. In some aspects, for each X the corresponding N_{c} is a distinct payload-encoding nucleic acid sequence. In some aspects, for each Y the corresponding U_{f} is a distinct universal MHC class II epitope-encoding nucleic acid sequence or a distinct MHC class II antigen-encoding nucleic nucleic acid sequence. The above payload encoding sequence formula in some instances only describes the portion of a cassette encoding concatenated payload sequences, such as concatenated T cell epitopes. For example, as an illustrative non-limiting example, in cassettes encoding concatenated T cell epitopes and one or more full-length SARS-CoV-2 proteins, the above payload encoding sequence formula describes the concatenated T cell epitopes and separately the cassette encodes one or more full-length SARS-CoV-2 proteins that are linked optionally using a multicistronic system, such as 2A ribosome skipping sequence elements (e.g., E2A, P2A, F2A, or T2A sequences) and/or an Internal Ribosome Entry Site (IRES) sequence elements.

In one example, elements present include where b=1, d=1, e=1, g=1, h=1, X=18, Y=2, and the vector backbone comprises a ChAdV68 vector, a=1, P is a CMV promoter, the at least one second poly(A) sequence is present, wherein the second poly(A) sequence is an exogenous poly(A) sequence to the vector backbone, and optionally wherein the exogenous poly(A) sequence comprises an SV40 poly(A) signal sequence or a BGH poly(A) signal sequence, and each N encodes a MHC class I epitope 7-15 amino acids in length, a MHC class II epitope, an epitope capable of stimulating a B cell response, or combinations thereof, L5 is a native 5′ linker sequence that encodes a native N-terminal amino acid sequence of the epitope, and wherein the 5′ linker sequence encodes a peptide that is at least 3 amino acids in length, L3 is a native 3′ linker sequence that encodes a native C-terminal amino acid sequence of the epitope, and wherein the 3′ linker sequence encodes a peptide that is at least 3 amino acids in length, and U is each of a PADRE class II sequence and a Tetanus toxoid MHC class II sequence. The above payload encoding sequence formula in some instances only describes the portion of a payload cassette encoding concatenated epitope sequences, such as concatenated T cell epitopes.

In one example, elements present include where b=1, d=1, e=1, g=1, h=1, X=18, Y=2, and the vector backbone comprises a Venezuelan equine encephalitis virus vector, a=0, and the payload cassette is operably linked to an endogenous 26S promoter, and the at least one polyadenylation poly(A) sequence is a poly(A) sequence of at least 80 consecutive A nucleotides provided by the backbone, and each N encodes a MHC class I epitope 7-15 amino acids in length, a MHC class II epitope, an epitope capable of stimulating a B cell response, or combinations thereof, L5 is a native 5′ linker sequence that encodes a native N-terminal amino acid sequence of the epitope, and wherein the 5′ linker sequence encodes a peptide that is at least 3 amino acids in length, L3 is a native 3′ linker sequence that encodes a native C-terminal amino acid sequence of the epitope, and wherein the 3′ linker sequence encodes a peptide that is at least 3 amino acids in length, and U is each of a PADRE class II sequence and a Tetanus toxoid MHC class II sequence.

The payload cassette can be described using the following formula to describe the ordered sequence of each element, from 5′ to 3′:

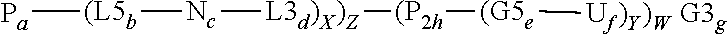

wherein P and P2 comprise promoter nucleotide sequences, N comprises an MHC class I epitope-encoding nucleic acid sequence, L5 comprises a 5′ linker sequence, L3 comprises a 3′ linker sequence, G5 comprises a nucleic acid sequences encoding an amino acid linker, G3 comprises one of the at least one nucleic acid sequences encoding an amino acid linker, U comprises an MHC class II antigen-encoding nucleic acid sequence, where for each X the corresponding Nc is an epitope encoding nucleic acid sequence, where for each Y the corresponding U_{f} is a MHC class II epitope-encoding nucleic acid sequence (e.g., universal MHC class II epitope-encoding nucleic acid sequence). A universal sequence can comprise at least one of Tetanus toxoid and PADRE. A universal sequence can comprise a Tetanus toxoid peptide. A universal sequence can comprise a PADRE peptide. A universal sequence can comprise a Tetanus toxoid and PADRE peptides. The composition and ordered sequence can be further defined by selecting the number of elements present, for example where a=0 or 1, where b=0 or 1, where c=1, where d=0 or 1, where e=0 or 1, where f=1, where g=0 or 1, where h=0 or 1, X=1 to 400, Y=0, 1, 2, 3, 4or5, Z=1to400, and W=0, 1, 2, 3, 4or5

In one example, elements present include where a=0, b=1, d=1, e=1, g=1, h=0, X=10, Y=2, Z=1, and W=1, describing where no additional promoter is present (e.g., only the promoter nucleotide sequence provided by a vector backbone, such as an RNA alphavirus or ChAdV backbone is present), 10 MHC class I epitopes are present, a 5′ linker is present for each N, a 3′ linker is present for each N, 2 MHC class II epitopes are present, a linker is present linking the two MHC class II epitopes, a linker is present linking the 5′ end of the two MHC class II epitopes to the 3′ linker of the final MHC class I epitope, and a linker is present linking the 3′ end of the two MHC class II epitopes to a vector backbone (e.g., a ChAdV or RNA alphavirus backbone).

Examples of linking the 3′ end of the cassette to a vector backbone (e.g., an RNA alphavirus backbone) include linking directly to the 3′ UTR elements provided by the vector backbone, such as a 3′ 19-nt CSE. Examples of linking the 5′ end of the cassette to a vector backbone (e.g., an RNA alphavirus backbone) include linking directly to a promoter or 5′ UTR element of the vector backbone, such as a subgenomic promoter sequence (e.g., a 26S subgenomic promoter sequence), an alphavirus 5′ UTR, a 51-nt CSE, or a 24-nt CSE.

Other examples include: where a=1 describing where a promoter other than the promoter nucleotide sequence provided by a vector backbone (e.g., a ChAdV or RNA alphavirus backbone) is present; where a=1 and Z is greater than 1 where multiple promoters other than the promoter nucleotide sequence provided by the vector backbone are present each driving expression of 1 or more distinct MHC class I epitope encoding nucleic acid sequences; where h=1 describing where a separate promoter is present to drive expression of the MHC class II epitope-encoding nucleic acid sequences; and where g=0 describing the MHC class II epitope-encoding nucleic acid sequence, if present, is directly linked to a vector backbone (e.g., a ChAdV or RNA alphavirus backbone). For example, a ChAdV vector backbone can have the cassette placed under the control of a CMV promoter/enhancer.

Other examples include where each MHC class I epitope that is present can have a 5′ linker, a 3′ linker, neither, or both. In examples where more than one MHC class I epitope is present in the same antigen cassette, some MHC class I epitopes may have both a 5′ linker and a 3′ linker, while other MHC class I epitopes may have either a 5′ linker, a 3′ linker, or neither. In other examples where more than one MHC class I epitope is present in the same antigen cassette, some MHC class I epitopes may have either a 5′ linker or a 3′ linker, while other MHC class I epitopes may have either a 5′ linker, a 3′ linker, or neither.

In examples where more than one MHC class II epitope is present in the same antigen cassette, some MHC class II epitopes may have both a 5′ linker and a 3′ linker, while other MHC class II epitopes may have either a 5′ linker, a 3′ linker, or neither. In other examples where more than one MHC class II epitope is present in the same antigen cassette, some MHC class II epitopes may have either a 5′ linker or a 3′ linker, while other MHC class II epitopes may have either a 5′ linker, a 3′ linker, or neither.

Other examples include where each payload that is present can have a 5′ linker, a 3′ linker, neither, or both. In examples where more than one payload is present in the same payload cassette, some payloads may have both a 5′ linker and a 3′ linker, while other payloads may have either a 5′ linker, a 3′ linker, or neither. In other examples where more than one payload is present in the same payload cassette, some payloads may have either a 5′ linker or a 3′ linker, while other payloads may have either a 5′ linker, a 3′ linker, or neither.

The promoter nucleotide sequences P and/or P2 can be the same as a promoter nucleotide sequence provided by a vector backbone, such as an RNA alphavirus backbone. For example, the promoter sequence provided by the RNA alphavirus backbone, Pn and P2, can each comprise a subgenomic promoter sequence (e.g., a 26S subgenomic promoter sequence) or a CMV promoter. The promoter nucleotide sequences P and/or P2 can be different from the promoter nucleotide sequence provided by a vector backbone (e.g., a ChAdV or RNA alphavirus backbone), as well as can be different from each other.

The 5′ linker L5 can be a native sequence or a non-natural sequence. Non-natural sequences include, but are not limited to, AAY, RR, and DPP. The 3′ linker L3 can also be a native sequence or a non-natural sequence. Additionally, L5 and L3 can both be native sequences, both be non-natural sequences, or one can be native and the other non-natural. For each X, the amino acid linkers can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length. For each X, the amino acid linkers can also be at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length.

For each X, the amino acid linkers can also be between 2-10, 2-15, 2-20, 2-25, 2-30, 2-40, 2-50, 3-10, 3-15, 3-20, 3-25, 3-30, 3-40, 3-50, 4-10, 4-15, 4-20, 4-25, 4-30, 4-40, 4-50, 5-10, 5-15, 5-20, 5-25, 5-30, 5-40, 5-50, 6-10, 6-15, 6-20, 6-25, 6-30, 6-40, 6-50, 7-10, 7-15, 7-20, 7-25, 7-30, 7-40, 7-50, 8-10, 8-15, 8-20, 8-25, 8-30, 8-40, or 8-50 amino acids in length.

The amino acid linker G5, for each Y, can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length. For each Y, the amino acid linkers can be also be at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length. G5 can also be between 2-10, 2-15, 2-20, 2-25, 2-30, 2-40, 2-50, 3-10, 3-15, 3-20, 3-25, 3-30, 3-40, 3-50, 4-10, 4-15, 4-20, 4-25, 4-30, 4-40, 4-50, 5-10, 5-15, 5-20, 5-25, 5-30, 5-40, 5-50, 6-10, 6-15, 6-20, 6-25, 6-30, 6-40, 6-50, 7-10, 7-15, 7-20, 7-25, 7-30, 7-40, 7-50, 8-10, 8-15, 8-20, 8-25, 8-30, 8-40, or 8-50 amino acids in length.

The amino acid linker G3 can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length. G3 can be also be at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length. G3 can also be between 2-10, 2-15, 2-20, 2-25, 2-30, 2-40, 2-50, 3-10, 3-15, 3-20, 3-25, 3-30, 3-40, 3-50, 4-10, 4-15, 4-20, 4-25, 4-30, 4-40, 4-50, 5-10, 5-15, 5-20, 5-25, 5-30, 5-40, 5-50, 6-10, 6-15, 6-20, 6-25, 6-30, 6-40, 6-50, 7-10, 7-15, 7-20, 7-25, 7-30, 7-40, 7-50, 8-10, 8-15, 8-20, 8-25, 8-30, 8-40, or 8-50 amino acids in length.

For each X, each N can encode a MHC class I epitope, a MHC class II epitope, an epitope/antigen capable of stimulating a B cell response, or a combination thereof. For each X, each N can encode a combination of a MHC class I epitope, a MHC class II epitope, and an epitope/antigen capable of stimulating a B cell response. For each X, each N can encode a combination of a MHC class I epitope and a MHC class II epitope. For each X, each N can encode a combination of a MHC class I epitope and an epitope/antigen capable of stimulating a B cell response. For each X, each N can encode a combination of a MHC class II epitope and an epitope/antigen capable of stimulating a B cell response. For each X, each N can encode a MHC class II epitope. For each X, each N can encode an epitope/antigen capable of stimulating a B cell response. For each X, each N can encode a MHC class I epitope 7-15 amino acids in length. For each X, each N can also encodes a MHC class I epitope 5, 6, 7, 8, 9, 10, 11, 12, 13, 14, 15, 16, 17, 18, 19, 20, 21, 22, 23, 24, 25, 26, 27, 28, 29, or 30 amino acids in length. For each X, each N can also encodes a MHC class I epitope at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length. For each X, each N can encode a MHC class II epitope. For each X, each N can encode an epitope capable of stimulating a B cell response.

A cassette, including each cassette respectively in a multicistronic system, can be at least 100, 200, 300, 400, 500, 600, 700, 800, or 900 nucleotides in length. A cassette can be at least 1000, 2000, 3000, 4000, 5000, 6000, 7000, 8000, 9000, or 10000 nucleotides in length. A cassette can be at least 1000 nucleotides in length. A cassette can be at least 2000 nucleotides in length. A cassette can be at least 3000 nucleotides in length. A cassette can be at least 4000 nucleotides in length. A cassette can be at least 5000 nucleotide s in length. A cassette can be at least 6000 nucleotides in length. A cassette can be at least 7000 nucleotides in length. A cassette can be at least 8000 nucleotides in length. A cassette can be at least 9000 nucleotides in length. A cassette can be between 100-1000, 100-2000, 100-3000, 100-4000, 100-5000, 100-6000, 100-7000, 100-8000, 100-9000, or 100-10000 nucleotides in length. A cassette can be between 500-1000, 500-2000, 500-3000, 500-4000, 500-5000, 500-6000, 500-7000, 500-8000, 500-9000, or 500-10000 nucleotides in length. A cassette can be between 1000-2000, 1000-3000, 1000-4000, 1000-5000, 1000-6000, 1000-7000, 1000-8000, 1000-9000, or 1000-10000 nucleotides in length. A cassette can be about the length deleted from an alphavirus (e.g., the length of deleted structural proteins in a VEE backbone). A cassette can be less than the length deleted from an alphavirus. A cassette can be more than the length deleted from an alphavirus.

For vectors including multiple cassettes, the total length of all cassettes combined can be at least 100, 200, 300, 400, 500, 600, 700, 800, or 900 nucleotides in length. For vectors including multiple cassettes, the total length of all cassettes combined can be at least 1000, 2000, 3000, 4000, 5000, 6000, 7000, 8000, 9000, or 10000 nucleotides in length For vectors including multiple cassettes, the total length of all cassettes combined can be between 100-1000, 100-2000, 100-3000, 100-4000, 100-5000, 100-6000, 100-7000, 100-8000, 100-9000, or 100-10000 nucleotides in length. For vectors including multiple cassettes, the total length of all cassettes combined can be between 500-1000, 500-2000, 500-3000, 500-4000, 500-5000, 500-6000, 500-7000, 500-8000, 500-9000, or 500-10000 nucleotides in length. For vectors including multiple cassettes, the total length of all cassettes combined can be between 1000-2000, 1000-3000, 1000-4000, 1000-5000, 1000-6000, 1000-7000, 1000-8000, 1000-9000, or 1000-10000 nucleotides in length.

A cassette can be 700 nucleotides or less. A cassette can be 700 nucleotides or less and encode 2 distinct epitope-encoding nucleic acid sequences (e.g., encode 2 distinct infectious disease or tumor derived nucleic acid sequences encoding an immunogenic polypeptide). A cassette can be 700 nucleotides or less and encode at least 2 distinct epitope-encoding nucleic acid sequences. A cassette can be 700 nucleotides or less and encode 3 distinct epitope-encoding nucleic acid sequences. A cassette can be 700 nucleotides or less and encode at least 3 distinct epitope-encoding nucleic acid sequences. A cassette can be 700 nucleotides or less and include 1-10, 1-5, 1, 2, 3, 4, 5, 6, 7, 8, 9, 10, or more payloads.

A cassette can be between 375-700 nucleotides in length. A cassette can be between 375-700 nucleotides in length and encode 2 distinct epitope-encoding nucleic acid sequences (e.g., encode 2 distinct infectious disease or tumor derived nucleic acid sequences encoding an immunogenic polypeptide). A cassette can be between 375-700 nucleotides in length and encode at least 2 distinct epitope-encoding nucleic acid sequences. A cassette can be between 375-700 nucleotides in length and encode 3 distinct epitope-encoding nucleic acid sequences. A cassette be between 375-700 nucleotides in length and encode at least 3 distinct epitope-encoding nucleic acid sequences. A cassette can be between 375-700 nucleotides in length and include 1-10, 1-5, 1, 2, 3, 4, 5, 6, 7, 8, 9, 10, or more payloads.

A cassette can be 600, 500, 400, 300, 200, or 100 nucleotides in length or less. A cassette can be 600, 500, 400, 300, 200, or 100 nucleotides in length or less and encode 2 distinct epitope-encoding nucleic acid sequences. A cassette can be 600, 500, 400, 300, 200, or 100 nucleotides in length or less and encode at least 2 distinct epitope-encoding nucleic acid sequences. A cassette can be 600, 500, 400, 300, 200, or 100 nucleotides in length or less and encode 3 distinct epitope-encoding nucleic acid sequences. A cassette can be 600, 500, 400, 300, 200, or 100 nucleotides in length or less and encode at least 3 distinct epitope-encoding nucleic acid sequences. A cassette can be 600, 500, 400, 300, 200, or 100 nucleotides in length or less and include 1-10, 1-5, 1, 2, 3, 4, 5, 6, 7, 8, 9, 10, or more payloads.

A cassette can be between 375-600, between 375-500, or between 375-400 nucleotides in length. A cassette can be between 375-600, between 375-500, or between 375-400 nucleotides in length and encode 2 distinct epitope-encoding nucleic acid sequences. A cassette can be between 375-600, between 375-500, or between 375-400 nucleotides in length and encode at least 2 distinct epitope-encoding nucleic acid sequences. A cassette can be between 375-600, between 375-500, or between 375-400 nucleotides in length and encode 3 distinct epitope-encoding nucleic acid sequences. A cassette can be between 375-600, between 375-500, or between 375-400 nucleotides in length and encode at least 3 distinct epitope-encoding nucleic acid sequences. A cassette can be between 375-600, between 375-500, or between 375-400 nucleotides in length and include 1-10, 1-5, 1, 2, 3, 4, 5, 6, 7, 8, 9, 10, or more payloads.

### Vaccine Compositions

A vaccine composition can further comprise an adjuvant and/or a carrier. Examples of useful adjuvants and carriers are given herein below. A composition can be associated with a carrier such as e.g., a protein or an antigen-presenting cell such as e.g. a dendritic cell (DC) capable of presenting the peptide to a T-cell.

Adjuvants are any substance whose admixture into a vaccine composition increases or otherwise modifies the immune response to a neoantigen. Carriers can be scaffold structures, for example a polypeptide or a polysaccharide, to which a neoantigen, is capable of being associated. Optionally, adjuvants are conjugated covalently or non-covalently.

The ability of an adjuvant to increase an immune response to an antigen is typically manifested by a significant or substantial increase in an immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th response into a primarily cellular, or Th response.

Suitable adjuvants include, but are not limited to 1018 ISS, alum, aluminium salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel vector system, PLG microparticles, resiquimod, SRL172, Virosomes and other Virus-like particles, YF-17D, VEGF trap, R848, beta-glucan, Pam3Cys, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Adjuvants such as incomplete Freund's or GM-CSF are useful. Several immunological adjuvants (e.g., MF59) specific for dendritic cells and their preparation have been described previously (Dupuis M, et al., Cell Immunol. 1998; 186(1):18-27; Allison A C; Dev Biol Stand. 1998; 92:3-11). Also cytokines can be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12) (Gabrilovich D I, et al., J Immunother Emphasis Tumor Immunol. 1996 (6):414-418).

CpG immunostimulatory oligonucleotides have also been reported to enhance the effects of adjuvants in a vaccine setting. Other TLR binding molecules such as RNA binding TLR 7, TLR 8 and/or TLR 9 may also be used.

Other examples of useful adjuvants include, but are not limited to, chemically modified CpGs (e.g. CpR, Idera), Poly(I:C)(e.g. polyi:CI2U), non-CpG bacterial DNA or RNA as well as immunoactive small molecules and antibodies such as cyclophosphamide, sunitinib, bevacizumab, celebrex, NCX-4016, sildenafil, tadalafil, vardenafil, sorafinib, XL-999, CP-547632, pazopanib, ZD2171, AZD2171, ipilimumab, tremelimumab, and SC58175, which may act therapeutically and/or as an adjuvant. The amounts and concentrations of adjuvants and additives can readily be determined by the skilled artisan without undue experimentation. Additional adjuvants include colony-stimulating factors, such as Granulocyte Macrophage Colony Stimulating Factor (GM-CSF, sargramostim).

A vaccine composition can comprise more than one different adjuvant. Furthermore, a therapeutic composition can comprise any adjuvant substance including any of the above or combinations thereof. It is also contemplated that a vaccine and an adjuvant can be administered together or separately in any appropriate sequence.

A carrier (or excipient) can be present independently of an adjuvant. The function of a carrier can for example be to increase the molecular weight of in particular mutant to increase activity or immunogenicity, to confer stability, to increase the biological activity, or to increase serum half-life. Furthermore, a carrier can aid presenting peptides to T-cells. A carrier can be any suitable carrier known to the person skilled in the art, for example a protein or an antigen presenting cell. A carrier protein could be but is not limited to keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier is generally a physiologically acceptable carrier acceptable to humans and safe. However, tetanus toxoid and/or diphtheria toxoid are suitable carriers. Alternatively, the carrier can be dextrans for example sepharose.

### Buffers

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g. HEPES), magnesium hydroxide, aluminum hydroxide, alginic acid, pyrogen-free water, isotonic saline, Ringer's solution, ethyl alcohol, and/or combinations thereof. Lubricating agents may selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

In some embodiments, a buffer is selected from the group consisting of citrate, succinate, malate, phosphate, histidine, glycine, MOPS, HEPES, Tris, and Bis-Tris. In some embodiments, a buffer is a citrate buffer. In some embodiments, a buffer is a succinate buffer. In some embodiments, a buffer is a malate buffer. In some embodiments, a buffer is a phosphate buffer. In some embodiments, a buffer is a Histidine buffer. In some embodiments, a buffer is MOPS. In some embodiments, a buffer is HEPES. In some embodiments, a buffer is Tris. In some embodiments, a buffer is Bis-Tris.

In some embodiments, a buffer has a salt concentration of 1-15 nM. In some embodiments, a buffer has a salt concentration of 3-13 nM. In some embodiments, a buffer has a salt concentration of 5-11 nM. In some embodiments, a buffer has a salt concentration of 6-10 nM. In some embodiments, a buffer has a salt concentration of 7-9 nM. In some embodiments, a buffer has a salt concentration of 7.5-8.5 nM. In some embodiments, a buffer has a salt concentration of 7.8-8.2 nM.

pH

In some embodiments, a buffer has a salt concentration of about 5.5 nM. In some embodiments, a buffer has a salt concentration of about 6.0 nM. In some embodiments, a buffer has a salt concentration of about 6.5 nM. In some embodiments, a buffer has a salt concentration of about 7.0 nM. In some embodiments, a buffer has a salt concentration of about 7.5 nM. In some embodiments, a buffer has a salt concentration of about 7.8 nM. In some embodiments, a buffer has a salt concentration of about 7.9 nM. In some embodiments, a buffer has a salt concentration of about 8.0 nM. In some embodiments, a buffer has a salt concentration of about 8.1 nM. In some embodiments, a buffer has a salt concentration of about 8.2 nM. In some embodiments, a buffer has a salt concentration of about 8.5 nM. In some embodiments, a buffer has a salt concentration of about 9.0 nM. In some embodiments, a buffer has a salt concentration of about 9.5 nM. In some embodiments, a buffer has a salt concentration of about 10.0 nM. In some embodiments, a buffer has a salt concentration of about 10.5 nM.

In some embodiments, a pharmaceutical composition has a pH of 6.0-9.2. In some embodiments, a pharmaceutical composition has a pH of 6.8-8.8. In some embodiments, a pharmaceutical composition has a pH of 7.0-8.6. In some embodiments, a pharmaceutical composition has a pH of 7.3-8.3. In some embodiments, a pharmaceutical composition has a pH of 7.4-8.2. In some embodiments, a pharmaceutical composition has a pH of 7.5-8.1. In some embodiments, a pharmaceutical composition has a pH of 7.6-8.0. In some embodiments, a pharmaceutical composition has a pH of 7.7-7.9.

In some embodiments, a pharmaceutical composition has a pH of about 5. In some embodiments, a pharmaceutical composition has a pH of about 5.5. In some embodiments, a pharmaceutical composition has a pH of about 6.0. In some embodiments, a pharmaceutical composition has a pH of about 7.0. In some embodiments, a pharmaceutical composition has a pH of about 7.5. In some embodiments, a pharmaceutical composition has a pH of about 7.6. In some embodiments, a pharmaceutical composition has a pH of about 7.7. In some embodiments, a pharmaceutical composition has a pH of about 7.8. In some embodiments, a pharmaceutical composition has a pH of about 7.9. In some embodiments, a pharmaceutical composition has a pH of about 8.0. In some embodiments, a pharmaceutical composition has a pH of about 8.1. In some embodiments, a pharmaceutical composition has a pH of 8.2. In some embodiments, a pharmaceutical composition has a pH of about 8.3. In some embodiments, a pharmaceutical composition has a pH of about 8.5. In some embodiments, a pharmaceutical composition has a pH of about 9.0. In some embodiments, a pharmaceutical composition has a pH of about 9.5.

### Cryoprotectants

In some embodiments, a cryoprotectant can be a compound used to protect the formulation from damage due to cold, for example, freezing. In some embodiments, a cryoprotectant can include a polyol, e.g., a carbohydrate, for example, sucrose, trehalose, glucose or a 2-hydroxypropyl-α-cyclodextrin. A sugar alcohol, such as sorbitol, can also be included in a cryoprotectant. In some embodiments, a cryoprotectant can include a protein, a peptide or an amino acid. For example, a cryoprotectant can include proline or hydroxyl proline. In some embodiments, an organic compound, such as glycerol, ethylene glycol, or propylene glycol, can be included in a cryoprotectant. In some instances, a cryoprotectant can include a polymer, for example, polyvinylpyrrolidone, polyethylene glycol or gelatin or hydroxyethylcellulose.

In some embodiments, a cryoprotectant is selected from the group consisting of ethanol, sucrose, maltose, lactose, glucose, galactose, trehalose, raffinose, other polyols and polyhydric alcohols. In some embodiments, a cryoprotectant is a carbohydrate. In some embodiments, a cryoprotectant is selected from the group consisting of sucrose, maltose, lactose, glucose, galactose, trehalose, and raffinose. In some embodiments, a cryoprotectant is sucrose. In some embodiments, a cryoprotectant is glucose. In some embodiments, a cryoprotectant is galactose. In some embodiments, a cryoprotectant is trehalose. In some embodiments, a cryoprotectant is raffinose.

In some embodiments, a pharmaceutical composition comprises 6-19 wt % cryoprotectant. In some embodiments, a pharmaceutical composition comprises 7-18 wt % cryoprotectant. In some embodiments, a pharmaceutical composition comprises 8-17 wt % cryoprotectant. In some embodiments, a pharmaceutical composition comprises 9-16 wt % cryoprotectant. In some embodiments, a pharmaceutical composition comprises 10-15 wt % cryoprotectant. In some embodiments, a pharmaceutical composition comprises 11-14 wt % cryoprotectant.

In some embodiments, a pharmaceutical composition comprises about 6 wt % cryoprotectant. In some embodiments, a pharmaceutical composition comprises about 7 wt % cryoprotectant. In some embodiments, a pharmaceutical composition comprises about 8 wt % cryoprotectant. In some embodiments, a pharmaceutical composition comprises about 9 wt % cryoprotectant. In some embodiments, a pharmaceutical composition comprises about 10 wt % cryoprotectant. In some embodiments, a pharmaceutical composition comprises about 11 wt % cryoprotectant. In some embodiments, a pharmaceutical composition comprises about 12 wt % cryoprotectant. In some embodiments, a pharmaceutical composition comprises about 13 wt % cryoprotectant. In some embodiments, a pharmaceutical composition comprises about 14 wt % cryoprotectant. In some embodiments, a pharmaceutical composition comprises about 15 wt % cryoprotectant. In some embodiments, a pharmaceutical composition comprises about 16 wt % cryoprotectant. In some embodiments, a pharmaceutical composition comprises about 17 wt % cryoprotectant. In some embodiments, a pharmaceutical composition comprises about 18 wt % cryoprotectant.

### Amino Acid

In some embodiments, an amino acid is selected from the group consisting of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. In some embodiments, an amino acid is selected from the group consisting of arginine, histidine, and lysine. In some embodiments, an amino acid is arginine. In some embodiments, an amino acid is histidine. In some embodiments, an amino acid is lysine.

In some embodiments, an amino acid has a concentration of 50-100 mM. In some embodiments, an amino acid has a concentration of 60-90 mM. In some embodiments, an amino acid has a concentration of 65-85 mM. In some embodiments, an amino acid has a concentration of 70-80 mM. In some embodiments, an amino acid has a concentration of 71-79 mM. In some embodiments, an amino acid has a concentration of 72-78 mM. In some embodiments, an amino acid has a concentration of 73-77 mM. In some embodiments, an amino acid has a concentration of 74-76 mM.

In some embodiments, an amino acid has a concentration of about 50 mM. In some embodiments, an amino acid has a concentration of about 55 mM. In some embodiments, an amino acid has a concentration of about 60 mM. In some embodiments, an amino acid has a concentration of about 65 mM. In some embodiments, an amino acid has a concentration of about 70 mM. In some embodiments, an amino acid has a concentration of about 71 mM. In some embodiments, an amino acid has a concentration of about 72 mM. In some embodiments, an amino acid has a concentration of about 73 mM. In some embodiments, an amino acid has a concentration of about 74 mM. In some embodiments, an amino acid has a concentration of about 75 mM. In some embodiments, an amino acid has a concentration of about 76 mM. In some embodiments, an amino acid has a concentration of about 77 mM. In some embodiments, an amino acid has a concentration of about 78 mM. In some embodiments, an amino acid has a concentration of about 79 mM. In some embodiments, an amino acid has a concentration of about 80 mM. In some embodiments, an amino acid has a concentration of about 85 mM. In some embodiments, an amino acid has a concentration of about 90 mM. In some embodiments, an amino acid has a concentration of about 95 mM. In some embodiments, an amino acid has a concentration of about 100 mM.

A vaccine can contain between 1 and 30 peptides, 2, 3, 4, 5, 6, 7, 8, 9, 10, 11, 12, 13, 14, 15, 16, 17, 18, 19, 20, 21, 22, 23, 24, 25, 26, 27, 28, 29, or 30 different peptides, 6, 7, 8, 9, 10 11, 12, 13, or 14 different peptides, or 12, 13 or 14 different peptides. Peptides can include post-translational modifications. A vaccine can contain between 1 and 100 or more nucleotide sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different nucleotide sequences, 6, 7, 8, 9, 10 11, 12, 13, or 14 different nucleotide sequences, or 12, 13 or 14 different nucleotide sequences. A vaccine can contain between 1 and 30 antigen sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different antigen sequences, 6, 7, 8, 9, 10 11, 12, 13, or 14 different antigen sequences, or 12, 13 or 14 different antigen sequences.

A vaccine can contain between 1 and 30 antigen-encoding nucleic acid sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different antigen-encoding nucleic acid sequences, 6, 7, 8, 9, 10 11, 12, 13, or 14 different antigen-encoding nucleic acid sequences, or 12, 13 or 14 different antigen-encoding nucleic acid sequences. Antigen-encoding nucleic acid sequences can refer to the antigen encoding portion of an antigen “cassette.” Features of an antigen cassette are described in greater detail herein. A cassette can contain two or more antigen-encoding nucleic acid sequences linked together in a cassette (e.g., concatenated antigen-encoding nucleic acid sequence encoding concatenated T cell epitopes).

A vaccine can contain between 1 and 30 distinct epitope-encoding nucleic acid sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more distinct epitope-encoding nucleic acid sequences, 6, 7, 8, 9, 10 11, 12, 13, or 14 distinct epitope-encoding nucleic acid sequences, or 12, 13 or 14 distinct epitope-encoding nucleic acid sequences. Epitope-encoding nucleic acid sequences can refer to sequences for individual epitope sequences, such as each of the concatenated T cell epitopes of two or more antigen-encoding nucleic acid sequences linked together in a cassette.

A vaccine can contain at least two repeats of an epitope-encoding nucleic acid sequence. A used herein, an “iteration” (or interchangeably a “repeat”) refers to two or more iterations of an identical nucleic acid epitope-encoding nucleic acid sequences (inclusive of the optional 5′ linker sequence and/or the optional 3′ linker sequences described herein) within an antigen-encoding nucleic acid sequence. In one example, the antigen-encoding nucleic acid sequence portion of a cassette encodes at least two iterations of an epitope-encoding nucleic acid sequence. In further non-limiting examples, the antigen-encoding nucleic acid sequence portion of a cassette encodes more than one distinct epitope, and at least one of the distinct epitopes is encoded by at least two iterations of the nucleic acid sequence encoding the distinct epitope (i.e., at least two distinct epitope-encoding nucleic acid sequences). In illustrative non-limiting examples, an antigen-encoding nucleic acid sequence encodes epitopes A, B, and C encoded by epitope-encoding nucleic acid sequences epitope-encoding sequence A (E_{A}), epitope-encoding sequence B (E_{B}), and epitope-encoding sequence C (E_{C}), and exemplary antigen-encoding nucleic acid sequences having iterations of at least one of the distinct epitopes are illustrated by, but is not limited to, the formulas below:
1. Iteration of one distinct epitope (iteration of epitope A):

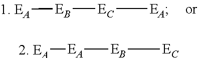

2. Iteration of multiple distinct epitopes (iterations of epitopes A, B, and C):

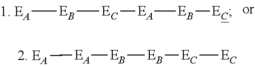

3. Multiple iterations of multiple distinct epitopes (iterations of epitopes A, B, and C):

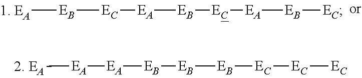

The above examples are not limiting and the antigen-encoding nucleic acid sequences having iterations of at least one of the distinct epitopes can encode each of the distinct epitopes in any order or frequency. For example, the order and frequency can be a random arrangement of the distinct epitopes, e.g., in an example with epitopes A, B, and C, by the formula E_{A}-E_{B}-E_{C}-E_{C}-E_{A}-E_{B}-E_{A}-E_{C}-E_{A}-E_{C}-E_{C}-E_{B}.

Also provided for herein is an antigen-encoding cassette, the antigen-encoding cassette having at least one antigen-encoding nucleic acid sequence described, from 5′ to 3′, by the formula:

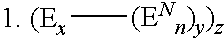

where E represents a nucleotide sequence including a distinct epitope-encoding nucleic acid sequence,
n represents the number of separate distinct epitope-encoding nucleic acid sequences and is any integer including 0,
E^{N} represents a nucleotide sequence comprising the separate distinct epitope-encoding nucleic acid sequence for each corresponding n,
for each iteration of z: x=0 or 1, y=0 or 1 for each n, and at least one of x or y=1, and
z=2 or greater, wherein the antigen-encoding nucleic acid sequence comprises at least two iterations of E, a given E^{N}, or a combination thereof.

Each E or E^{N} can independently comprise any epitope-encoding nucleic acid sequence described herein (e.g., a peptide encoding an infectious disease T cell epitope and/or a neoantigen epitope). For example, Each E or E^{N} can independently comprises a nucleotide sequence described, from 5′ to 3′, by the formula (L5_{b}-N_{c}-L3_{d}), where N comprises the distinct epitope-encoding nucleic acid sequence associated with each E or E^{N}, where c=1, L5 comprises a 5′ linker sequence, where b=0 or 1, and L3 comprises a 3′ linker sequence, where d=0 or 1. Epitopes and linkers that can be used are further described herein, e.g., see V.A. Antigen Cassette.

Iterations of an epitope-encoding nucleic acid sequences (inclusive of optional 5′ linker sequence and/or the optional 3′ linker sequences) can be linearly linked directly to one another (e.g., E_{A}-E_{A}- . . . as illustrated above). Iterations of an epitope-encoding nucleic acid sequences can be separated by one or more additional nucleotides sequences. In general, iterations of an epitope-encoding nucleic acid sequences can be separated by any size nucleotide sequence applicable for the compositions described herein. In one example, iterations of an epitope-encoding nucleic acid sequences can be separated by a separate distinct epitope-encoding nucleic acid sequence (e.g., E_{A}-E_{B}-E_{C}-E_{A} . . . , as illustrated above). In examples where iterations are separated by a single separate distinct epitope-encoding nucleic acid sequence, and each epitope-encoding nucleic acid sequences (inclusive of optional 5′ linker sequence and/or the optional 3′ linker sequences) encodes a peptide 25 amino acids in length, the iterations can be separated by 75 nucleotides, such as in antigen-encoding nucleic acid represented by E_{A}-E_{B}-E_{A} . . . , E_{A} is separated by 75 nucleotides. In an illustrative example, an antigen-encoding nucleic acid having the sequence VTNTEMFVTAPDNLGYMYEVQWPGQTQPQIANCSVYDFFVWLHYYSVRD TVTNTEMFVTAPDNLGYMYEVQWPGQTQPQIANCSVYDFFVWLHYYSVRDT (SEQ ID NO: 2) encoding iterations of 25mer antigens Trp1 (VTNTEMFVTAPDNLGYMYEVQWPGQ (SEQ ID NO: 3)) and Trp2 (TQPQIANCSVYDFFVWLHYYSVRDT (SEQ ID NO: 4)), the iterations of Trp1 are separated by the 25mer Trp2 and thus the iterations of the Trp1 epitope-encoding nucleic acid sequences are separated the 75 nucleotide Trp2 epitope-encoding nucleic acid sequence. In examples where iterations are separated by 2, 3, 4, 5, 6, 7, 8, or 9 separate distinct epitope-encoding nucleic acid sequence, and each epitope-encoding nucleic acid sequences (inclusive of optional 5′ linker sequence and/or the optional 3′ linker sequences) encodes a peptide 25 amino acids in length, the iterations can be separated by 150, 225, 300, 375, 450, 525, 600, or 675 nucleotides, respectively.

In one embodiment, different peptides and/or polypeptides or nucleotide sequences encoding them are selected so that the peptides and/or polypeptides capable of associating with different MHC molecules, such as different MHC class I molecules and/or different MHC class II molecules. In some aspects, one vaccine composition comprises coding sequence for peptides and/or polypeptides capable of associating with the most frequently occurring MHC class I molecules and/or different MHC class II molecules. Hence, vaccine compositions can comprise different fragments capable of associating with at least 2 preferred, at least 3 preferred, or at least 4 preferred MHC class I molecules and/or different MHC class II molecules.

The vaccine composition can be capable of stimulating a specific cytotoxic T-cell response and/or a specific helper T-cell response. The vaccine composition can be capable of stimulating a specific cytotoxic T-cell response and a specific helper T-cell response.

The vaccine composition can be capable of stimulating a specific B-cell response (e.g., an antibody response).

The vaccine composition can be capable of stimulating a specific cytotoxic T-cell response, a specific helper T-cell response, and/or a specific B-cell response. The vaccine composition can be capable of stimulating a specific cytotoxic T-cell response and a specific B-cell response. The vaccine composition can be capable of stimulating a specific helper T-cell response and a specific B-cell response. The vaccine composition can be capable of stimulating a specific cytotoxic T-cell response, a specific helper T-cell response, and a specific B-cell response.

A vaccine composition can further comprise an adjuvant and/or a carrier. Examples of useful adjuvants and carriers are given herein below. A composition can be associated with a carrier such as e.g. a protein or an antigen-presenting cell such as, e.g., a dendritic cell (DC) capable of presenting the peptide to a T-cell.

Adjuvants are any substance whose admixture into a vaccine composition increases or otherwise modifies the immune response to an antigen. Carriers can be scaffold structures, for example a polypeptide or a polysaccharide, to which an antigen, is capable of being associated. Optionally, adjuvants are conjugated covalently or non-covalently.

The ability of an adjuvant to increase an immune response to an antigen is typically manifested by a significant or substantial increase in an immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th response into a primarily cellular, or Th response.

Suitable adjuvants include, but are not limited to 1018 ISS, alum, aluminium salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel vector system, PLG microparticles, resiquimod, SRL172, Virosomes and other Virus-like particles, YF-17D, VEGF trap, R848, beta-glucan, Pam3Cys, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Adjuvants such as incomplete Freund's or GM-CSF are useful. Several immunological adjuvants (e.g., MF59) specific for dendritic cells and their preparation have been described previously (Dupuis M, et al., Cell Immunol. 1998; 186(1):18-27; Allison A C; Dev Biol Stand. 1998; 92:3-11). Also cytokines can be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12) (Gabrilovich D I, et al., J Immunother Emphasis Tumor Immunol. 1996 (6):414-418).

CpG immunostimulatory oligonucleotides have also been reported to enhance the effects of adjuvants in a vaccine setting. Other TLR binding molecules such as RNA binding TLR 7, TLR 8 and/or TLR 9 may also be used.

Other examples of useful adjuvants include, but are not limited to, chemically modified CpGs (e.g. CpR, Idera), Poly(I:C)(e.g. polyi:CI2U), non-CpG bacterial DNA or RNA as well as immunoactive small molecules and antibodies such as cyclophosphamide, sunitinib, bevacizumab, celebrex, NCX-4016, sildenafil, tadalafil, vardenafil, sorafinib, XL-999, CP-547632, pazopanib, ZD2171, AZD2171, ipilimumab, tremelimumab, and SC58175, which may act therapeutically and/or as an adjuvant. The amounts and concentrations of adjuvants and additives can readily be determined by the skilled artisan without undue experimentation. Additional adjuvants include colony-stimulating factors, such as Granulocyte Macrophage Colony Stimulating Factor (GM-CSF, sargramostim).

A vaccine composition can comprise more than one different adjuvant. Furthermore, a therapeutic composition can comprise any adjuvant substance including any of the above or combinations thereof. It is also contemplated that a vaccine and an adjuvant can be administered together or separately in any appropriate sequence.

A carrier (or excipient) can be present independently of an adjuvant. The function of a carrier can for example be to increase the molecular weight of in particular mutant to increase activity or immunogenicity, to confer stability, to increase the biological activity, or to increase serum half-life. Furthermore, a carrier can aid presenting peptides to T-cells. A carrier can be any suitable carrier known to the person skilled in the art, for example a protein or an antigen presenting cell. A carrier protein could be but is not limited to keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier is generally a physiologically acceptable carrier acceptable to humans and safe. However, tetanus toxoid and/or diphtheria toxoid are suitable carriers. Alternatively, the carrier can be dextrans for example sepharose.

Cytotoxic T-cells (CTLs) recognize an antigen in the form of a peptide bound to an MHC molecule rather than the intact foreign antigen itself. The MHC molecule itself is located at the cell surface of an antigen presenting cell. Thus, an activation of CTLs is possible if a trimeric complex of peptide antigen, MHC molecule, and APC is present. Correspondingly, it may enhance the immune response if not only the peptide is used for activation of CTLs, but if additionally APCs with the respective MHC molecule are added. Therefore, in some embodiments a vaccine composition additionally contains at least one antigen presenting cell.

Antigens can also be included in viral vector-based vaccine platforms, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In Vivo Gene Delivery, J. Virol. (1998) 72 (12): 9873-9880). Dependent on the packaging capacity of the above mentioned viral vector-based vaccine platforms, this approach can deliver one or more nucleotide sequences that encode one or more antigen peptides. The sequences may be flanked by non-mutated sequences, may be separated by linkers or may be preceded with one or more sequences targeting a subcellular compartment (See, e.g., Gros et al., Prospective identification of neoantigen-specific lymphocytes in the peripheral blood of melanoma patients, Nat Med. (2016) 22 (4):433-8, Stronen et al., Targeting of cancer neoantigens with donor-derived T cell receptor repertoires, Science. (2016) 352 (6291):1337-41, Lu et al., Efficient identification of mutated cancer antigens recognized by T cells associated with durable tumor regressions, Clin Cancer Res. (2014) 20 (13):3401-10). Upon introduction into a host, infected cells express the antigens, and thereby stimulate a host immune (e.g., CTL) response against the peptide(s). Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vaccine vectors useful for therapeutic administration or immunization of antigens, e.g., Salmonella typhi vectors, and the like will be apparent to those skilled in the art from the description herein.

### Additional Considerations for Vaccine Design and Manufacture

Determination of a Set of Peptides that Cover all Tumor Subclones

Truncal peptides, meaning those presented by all or most tumor subclones, can be prioritized for inclusion into a vaccine. Optionally, if there are no truncal peptides predicted to be presented and immunogenic with high probability, or if the number of truncal peptides predicted to be presented and immunogenic with high probability is small enough that additional non-truncal peptides can be included in the vaccine, then further peptides can be prioritized by estimating the number and identity of tumor subclones and choosing peptides so as to maximize the number of tumor subclones covered by a vaccine.

### Antigen Prioritization

After all of the above antigen filters are applied, more candidate antigens may still be available for vaccine inclusion than the vaccine technology can support. Additionally, uncertainty about various aspects, of the antigen analysis may remain and tradeoffs may exist between different properties of candidate vaccine antigens. Thus, in place of predetermined filters at each step of the selection process, an integrated multi-dimensional model can be considered that places candidate antigens in a space with at least the following axes and optimizes selection using an integrative approach.

Risk of auto-immunity or tolerance (risk of germline) (lower risk of auto-immunity is typically preferred)

Probability of sequencing artifact (lower probability of artifact is typically preferred)

Probability of immunogenicity (higher probability of immunogenicity is typically preferred)

Probability of presentation (higher probability of presentation is typically preferred)

Gene expression (higher expression is typically preferred)

Coverage of HLA genes (larger number of HLA molecules involved in the presentation of a set of antigens may lower the probability that a tumor, an infectious disease, and/or an infected cell will escape immune attack via downregulation or mutation of HLA molecules)

Coverage of HLA classes (covering both HLA-I and HLA-II may increase the probability of therapeutic response and decrease the probability of tumor or infectious disease escape)

Additionally, optionally, antigens can be deprioritized (e.g., excluded) from the vaccination if they are predicted to be presented by HLA alleles lost or inactivated in either all or part of the patient's tumor or infected cell. HLA allele loss can occur by either somatic mutation, loss of heterozygosity, or homozygous deletion of the locus. Methods for detection of HLA allele somatic mutation are well known in the art, e.g. (Shukla et al., 2015). Methods for detection of somatic LOH and homozygous deletion (including for HLA locus) are likewise well described. (Carter et al., 2012; McGranahan et al., 2017; Van Loo et al., 2010). Antigens can also be deprioritized if mass-spectrometry data indicates a predicted antigen is not presented by a predicted HLA allele.

### Self-Amplifying RNA Vectors

In general, all self-amplifying RNA (SAM) vectors contain a self-amplifying backbone derived from a self-replicating virus. The term “self-amplifying backbone” refers to minimal sequence(s) of a self-replicating virus that allows for self-replication of the viral genome. For example, minimal sequences that allow for self-replication of an alphavirus can include conserved sequences for nonstructural protein-mediated amplification (e.g., a nonstructural protein 1 (nsP1) gene, a nsP2 gene, a nsP3 gene, a nsP4 gene, and/or a polyA sequence). A self-amplifying backbone can also include sequences for expression of subgenomic viral RNA (e.g., a 26S promoter element for an alphavirus). samRNA vectors can be positive-sense RNA polynucleotides or negative-sense RNA polynucleotides, such as vectors with backbones derived from positive-sense or negative-sense self-replicating viruses. Self-replicating viruses include, but are not limited to, alphaviruses, flaviviruses (e.g., Kunjin virus), measles viruses, and rhabdoviruses (e.g., rabies virus and vesicular stomatitis virus). Examples of samRNA vector systems derived from self-replicating viruses are described in greater detail in Lundstrom (Molecules. 2018 Dec. 13; 23(12). pii: E3310. doi: 10.3390/molecules23123310), herein incorporated by reference for all purposes.

### Alphavirus Biology

Alphaviruses are members of the family Togaviridae, and are positive-sense single stranded RNA viruses. Members are typically classified as either Old World, such as Sindbis, Ross River, Mayaro, Chikungunya, and Semliki Forest viruses, or New World, such as eastern equine encephalitis, Aura, Fort Morgan, or Venezuelan equine encephalitis virus and its derivative strain TC-83 (Strauss Microbial Review 1994). A natural alphavirus genome is typically around 12 kb in length, the first two-thirds of which contain genes encoding non-structural proteins (nsPs) that form RNA replication complexes for self-replication of the viral genome, and the last third of which contains a subgenomic expression cassette encoding structural proteins for virion production (Frolov RNA 2001).

A model lifecycle of an alphavirus involves several distinct steps (Strauss Microbiol Review 1994, Jose Future Microbiol 2009). Following virus attachment to a host cell, the virion fuses with membranes within endocytic compartments resulting in the eventual release of genomic RNA into the cytosol. The genomic RNA, which is in a plus-strand orientation and comprises a 5′ methylguanylate cap and 3′ polyA tail, is translated to produce non-structural proteins nsP1-4 that form the replication complex. Early in infection, the plus-strand is then replicated by the complex into a minus-stand template. In the current model, the replication complex is further processed as infection progresses, with the resulting processed complex switching to transcription of the minus-strand into both full-length positive-strand genomic RNA, as well as the 26S subgenomic positive-strand RNA containing the structural genes. Several conserved sequence elements (CSEs) of alphavirus have been identified to potentially play a role in the various RNA replication steps including; a complement of the 5′ UTR in the replication of plus-strand RNAs from a minus-strand template, a 51-nt CSE in the replication of minus-strand synthesis from the genomic template, a 24-nt CSE in the junction region between the nsPs and the 26S RNA in the transcription of the subgenomic RNA from the minus-strand, and a 3′ 19-nt CSE in minus-strand synthesis from the plus-strand template.

Following the replication of the various RNA species, virus particles are then typically assembled in the natural lifecycle of the virus. The 26S RNA is translated and the resulting proteins further processed to produce the structural proteins including capsid protein, glycoproteins E1 and E2, and two small polypeptides E3 and 6K (Strauss 1994). Encapsidation of viral RNA occurs, with capsid proteins normally specific for only genomic RNA being packaged, followed by virion assembly and budding at the membrane surface.

### Alphavirus as a Delivery Vector

Alphaviruses (including alphavirus sequences, features, and other elements) can be used to generate alphavirus-based delivery vectors (also be referred to as alphavirus vectors, alphavirus viral vectors, alphavirus vaccine vectors, self-replicating RNA (srRNA) vectors, or self-amplifying mRNA (SAM) vectors). Alphaviruses have previously been engineered for use as expression vector systems (Pushko 1997, Rheme 2004). Alphaviruses offer several advantages, particularly in a vaccine setting where heterologous antigen expression can be desired. Due to its ability to self-replicate in the host cytosol, alphavirus vectors are generally able to produce high copy numbers of the expression cassette within a cell resulting in a high level of heterologous antigen production. Additionally, the vectors are generally transient, resulting in improved biosafety as well as reduced induction of immunological tolerance to the vector. The public, in general, also lacks pre-existing immunity to alphavirus vectors as compared to other standard viral vectors, such as human adenovirus. Alphavirus based vectors also generally result in cytotoxic responses to infected cells. Cytotoxicity, to a certain degree, can be important in a vaccine setting to properly stimulate an immune response to the heterologous antigen expressed. However, the degree of desired cytotoxicity can be a balancing act, and thus several attenuated alphaviruses have been developed, including the TC-83 strain of VEE. Thus, an example of an antigen expression vector described herein can utilize an alphavirus backbone that allows for a high level of antigen expression, stimulates a robust immune response to antigen, does not stimulate an immune response to the vector itself, and can be used in a safe manner. Furthermore, the antigen expression cassette can be designed to stimulate different levels of an immune response through optimization of which alphavirus sequences the vector uses, including, but not limited to, sequences derived from VEE or its attenuated derivative TC-83.

Several expression vector design strategies have been engineered using alphavirus sequences (Pushko 1997). In one strategy, an alphavirus vector design includes inserting a second copy of the 26S promoter sequence elements downstream of the structural protein genes, followed by a heterologous gene (Frolov 1993). Thus, in addition to the natural non-structural and structural proteins, an additional subgenomic RNA is produced that expresses the heterologous protein. In this system, all the elements for production of infectious virions are present and, therefore, repeated rounds of infection of the expression vector in non-infected cells can occur.

Another expression vector design makes use of helper virus systems (Pushko 1997). In this strategy, the structural proteins are replaced by a heterologous gene. Thus, following self-replication of viral RNA mediated by still intact non-structural genes, the 26S subgenomic RNA provides for expression of the heterologous protein. Traditionally, additional vectors that expresses the structural proteins are then supplied in trans, such as by co-transfection of a cell line, to produce infectious virus. A system is described in detail in U.S. Pat. No. 8,093,021, which is herein incorporated by reference in its entirety, for all purposes. The helper vector system provides the benefit of limiting the possibility of forming infectious particles and, therefore, improves biosafety. In addition, the helper vector system reduces the total vector length, potentially improving the replication and expression efficiency. Thus, an example of an antigen expression vector described herein can utilize an alphavirus backbone wherein the structural proteins are replaced by an antigen cassette, the resulting vector both reducing biosafety concerns, while at the same time promoting efficient expression due to the reduction in overall expression vector size.

### Self-Amplifying Virus Production In Vitro

A convenient technique well-known in the art for RNA production is in vitro transcription (IVT). In this technique, a DNA template of the desired vector is first produced by techniques well-known to those in the art, including standard molecular biology techniques such as cloning, restriction digestion, ligation, gene synthesis (e.g., chemical and/or enzymatic synthesis), and polymerase chain reaction (PCR).

The DNA template contains an RNA polymerase promoter at the 5′ end of the sequence desired to be transcribed into RNA (e.g., SAM). Promoters include, but are not limited to, bacteriophage polymerase promoters such as T3, T7, K11, or SP6. Depending on the specific RNA polymerase promoter sequence chosen, additional 5′ nucleotides can be transcribed in addition to the desired sequence. For example, the canonical T7 promoter can be referred to by the sequence TAATACGACTCACTATAGG (SEQ ID NO: 5), in which an IVT reaction using the DNA template TAATACGACTCACTATAGGN (SEQ ID NO: 6) for the production of desired sequence N will result in the mRNA sequence GG-N. In general, and without wishing to be bound by theory, T7 polymerase more efficiently transcribes RNA transcripts beginning with guanosine. In instances where additional 5′ nucleotides are not desired (e.g., no additional GG), the RNA polymerase promoter contained in the DNA template can be a sequence the results in transcripts containing only the 5′ nucleotides of the desired sequence, e.g., a samRNA having the native 5′ sequence of the self-replicating virus from which the samRNA vector is derived. For example, a minimal T7 promoter can be referred to by the sequence TAATACGACTCACTATA (SEQ ID NO: 7), in which an IVT reaction using the DNA template TAATACGACTCACTATAN (SEQ ID NO: 8) for the production of desired sequence N will result in the mRNA sequence N. Likewise, a minimal SP6 promoter referred to by the sequence ATTTAGGTGACACTATA (SEQ ID NO: 9) can be used to generate transcripts without additional 5′ nucleotides. In a typical IVT reaction, the DNA template is incubated with the appropriate RNA polymerase enzyme, buffer agents, and nucleotides (NTPs).

The resulting RNA polynucleotide can optionally be further modified including, but limited to, addition of a 5′ cap structure such as 7-methyl guanosine or a related structure, and optionally modifying the 3′ end to include a polyadenylate (polyA) tail. In a modified IVT reaction, RNA is capped with a 5′ cap structure co-transcriptionally through the addition of cap analogues during IVT. Cap analogues can include dinucleotide (m⁷G-ppp-N) cap analogues or trinucleotide (m⁷G-ppp-N-N) cap analogues, where N represents a nucleotide or modified nucleotide (e.g., ribonucleosides including, but not limited to, adenosine, guanosine, cytidine, and uridine). Exemplary cap analogues and their use in IVT reactions are also described in greater detail in U.S. Pat. No. 10,519,189, herein incorporated by reference for all purposes. As discussed, T7 polymerase more efficiently transcribes RNA transcripts beginning with guanosine. To improve transcription efficiency in templates that do not begin with guanosine, a trinucleotide cap analogue (m⁷G-ppp-N-N) can be used. The trinucleotide cap analogue can increase transcription efficiency 2, 3, 4, 5, 6, 7, 8, 9, 10, 11, 12, 13, 14, 15, 16, 17, 18, 19, 20-fold or more relative to an IVT reaction using a dinucleotide cap analogue (m⁷G-ppp-N).

A 5′ cap structure can also be added following transcription, such as using a vaccinia capping system (e.g., NEB Cat. No. M2080) containing mRNA 2′-O-methyltransferase and S-Adenosyl methionine.

The resulting RNA polynucleotide can optionally be further modified separately from or in addition to the capping techniques described including, but limited to, modifying the 3′ end to include a polyadenylate (polyA) tail.

The RNA can then be purified using techniques well-known in the field, such as phenol-chloroform extraction or column purification (e.g., chromatography-based purification).

### Delivery Via Lipid Nanoparticle

An important aspect to consider in vaccine vector design is immunity against the vector itself (Riley 2017). This may be in the form of preexisting immunity to the vector itself, such as with certain human adenovirus systems, or in the form of developing immunity to the vector following administration of the vaccine. The latter is an important consideration if multiple administrations of the same vaccine are performed, such as separate priming and boosting doses, or if the same vaccine vector system is to be used to deliver different antigen cassettes.

In the case of alphavirus vectors, the standard delivery method is the previously discussed helper virus system that provides capsid, E1, and E2 proteins in trans to produce infectious viral particles. However, it is important to note that the E1 and E2 proteins are often major targets of neutralizing antibodies (Strauss 1994). Thus, the efficacy of using alphavirus vectors to deliver antigens of interest to target cells may be reduced if infectious particles are targeted by neutralizing antibodies.

An alternative to viral particle mediated gene delivery is the use of nanomaterials to deliver expression vectors (Riley 2017). Nanomaterial vehicles, importantly, can be made of non-immunogenic materials and generally avoid stimulating immunity to the delivery vector itself. These materials can include, but are not limited to, lipids, inorganic nanomaterials, and other polymeric materials. Lipids can be cationic, anionic, or neutral. The materials can be synthetic or naturally derived, and in some instances biodegradable. Lipids can include fats, cholesterol, phospholipids, lipid conjugates including, but not limited to, polyethyleneglycol (PEG) conjugates (PEGylated lipids), waxes, oils, glycerides, and fat soluble vitamins.

Lipid nanoparticles (LNPs) are an attractive delivery system due to the amphiphilic nature of lipids enabling formation of membranes and vesicle like structures (Riley 2017). In general, these vesicles deliver the expression vector by absorbing into the membrane of target cells and releasing nucleic acid into the cytosol. In addition, LNPs can be further modified or functionalized to facilitate targeting of specific cell types. Another consideration in LNP design is the balance between targeting efficiency and cytotoxicity. Lipid compositions generally include defined mixtures of cationic, neutral, anionic, and amphipathic lipids. In some instances, specific lipids are included to prevent LNP aggregation, prevent lipid oxidation, or provide functional chemical groups that facilitate attachment of additional moieties. Lipid composition can influence overall LNP size and stability. In an example, the lipid composition comprises dilinoleylmethyl-4-dimethylaminobutyrate (MC3) or MC3-like molecules. MC3 and MC3-like lipid compositions can be formulated to include one or more other lipids, such as a PEG or PEG-conjugated lipid, a sterol, or neutral lipids.

Nucleic-acid vectors, such as expression vectors, exposed directly to serum can have several undesirable consequences, including degradation of the nucleic acid by serum nucleases or off-target stimulation of the immune system by the free nucleic acids. Therefore, encapsulation of the alphavirus vector can be used to avoid degradation, while also avoiding potential off-target effects. In certain examples, an alphavirus vector is fully encapsulated within the delivery vehicle, such as within the aqueous interior of an LNP. Encapsulation of the alphavirus vector within an LNP can be carried out by techniques well-known to those skilled in the art, such as microfluidic mixing and droplet generation carried out on a microfluidic droplet generating device. Such devices include, but are not limited to, standard T-junction devices or flow-focusing devices. In an example, the desired lipid formulation, such as MC3 or MC3-like containing compositions, is provided to the droplet generating device in parallel with the alphavirus delivery vector and other desired agents, such that the delivery vector and desired agents are fully encapsulated within the interior of the MC3 or MC3-like based LNP. In an example, the droplet generating device can control the size range and size distribution of the LNPs produced. For example, the LNP can have a size ranging from 1 to 1000 nanometers in diameter, e.g., 1, 10, 50, 100, 500, or 1000 nanometers. Following droplet generation, the delivery vehicles encapsulating the expression vectors can be further treated or modified to prepare them for administration.

Conventional and pharmaceutically acceptable routes of administration include, but are not limited to, intranasal, intramuscular, intratracheal, subcutaneous, intradermal, rectal, oral and other parental routes of administration. Routes of administration may be combined, if desired, or adjusted depending upon the immunogen or the disease. For example, in prophylaxis of rabies, the subcutaneous, intratracheal and intranasal routes are preferred. The route of administration primarily will depend on the nature of the disease being treated.

The levels of immunity to antigen(s) can be monitored to determine the need, if any, for boosters. Following an assessment of antibody titers in the serum, for example, optional booster immunizations may be desired.

### Exemplification

### Example 1: Short Term Stability for samRNA-LNP DP in Novel Formulations Vs Current TSM Formulation

Previously, self-amplifying RNA encapsulated in lipid nanoparticles (samRNA-LNP)-based drug products (DP) were stored formulated in TSM (5 mM Tris, 10 wt % Sucrose and 10 wt % Maltose) buffer and were shown to be stable for long term storage at ≤−60° C. Due to limited clinical sites with ≤−60° C. storage capability, it is desired to test the stability of samRNA-LNP DP in other potential formulations and at alternative temperature conditions that may enable distribution to clinical sites that do not have ≤−60° C. storage capability.

The samRNA-LNP DPs stored in TSM are unstable when stored at temperatures above −60° C. and particles tend to increase in hydrodynamic size, exhibit time and temperature dependent degradation in the full-length profile of the encapsulated samRNA and concurrent loss in cellular potency. These observed undesirable changes in critical quality attributes in the DP when stored at temperatures above −60° C. in the current TSM formulation make storage of DPs at temperatures above −60° C. unsuitable for clinical administration. To find a formulation that would be stable at storage temperatures above −60° C., two studies were assessed: Short-Term pH and Excipient Scouting Study and a Short Term Robustness of Stability Study.

The studies included buffer exchanging samRNA-LNP DPs into investigation buffer systems via Tangential Flow Filtration (99.9% Buffer exchanged) and assessing the stability of the DPs at various timepoints and temperatures in investigational buffer systems as compared to the current TSM buffer system. The two studies were as follows:

Short-Term pH and Excipient Scouting Study: samRNA-LNP construct stability at pH 5.5 to pH 8.0 using DP in TSM buffer as a control. The investigational buffer formulations were:
mod-GB6: 25 mM Succinic Acid, 12 wt % Sucrose, 100 mM NaCl, 50 mM ArgHCl (pH 5.80)
mod-GB7: 10 mM Citric Acid, 15 wt % Sucrose, 50 mM ArgHCl (pH 5.5)
mod-GB8: 20 mM Histidine, 15 wt % Sucrose, 100 mM NaCl, 50 mM ArgHCl (pH 6.1)
mod-TSM1: 5 mM Tris, 15 wt % Sucrose, 100 mM NaCl (pH 7.8)
TSA-2: 5 mM Tris, 15 wt % Sucrose, 50 mM ArgHCl (pH 7.8)

Short Term Robustness of Stability Study: samRNA-LNP construct stability was assessed in the lead formulation buffer candidate from the preceding study along with the stability of the samRNA DP in a modified formulation of the lead formulation candidate from the preceding study (excipient components were varied over a narrow range to establish robustness and further refine the selection of the most optimized formulation).

### Example 2: Short-Term pH and Excipient Scouting Study Using samRNA DP

Initially, the formulations (pH 5.5 to 8.0) were assessed for short term (3 month) stability to determine the optimum pH for the formulation matrix. The efficacy of the formulation was assessed at various timepoints and at various temperatures, including an accelerated storage conditions of 5° C., 25° C. and a stressed storage condition of 40° C. The results of the short-term stability of study as assessed by Full Length profile by capillary electrophoresis, concentration and encapsulation determination by absorbance spectroscopy, and size and polydispersity determination by Dynamic Light scattering is captured in the following graphs.

The formulation composition, pH and osmolality of DP in various investigational matrix is outlined in Table 1 as follows:

TABLE 1

| | | | Osmo |
|---|---|---|---|
| | | | (mOSm/ |
| Reference | Formulation Matrix | pH | Kg) |
| mod GB-6 | 25 mM Succinic Acid, 12 wt % Sucrose, 100 | 5.8 | 823 |
| | mM NaCl, 50 mM ArgHCl | | |
| mod GB-7 | 10 mM Citric Acid, 15 wt % Sucrose, 50 | 5.5 | 711 |
| | mM Arginine HCl | | |
| mod GB-8 | 20 mM Histidine, 15 wt % Sucrose, 100 mM | 6.1 | 1009 |
| | NaCl, 50 mM Arginine HCl | | |
| mod | 5 mM Tris, 15 wt % Sucrose, 100 mM NaCl | 7.8 | 754 |
| TSM-1 | | | |
| TSA-2 | 5 mM Tris, 15 wt % Sucrose, 50 mM | 7.8 | 587 |
| | Arginine | | |
| TSM | 5 mM Tris, 10 wt % Sucrose and 10% | 8 | 780 |
| | Maltose at 0.06 mg/mL | | |

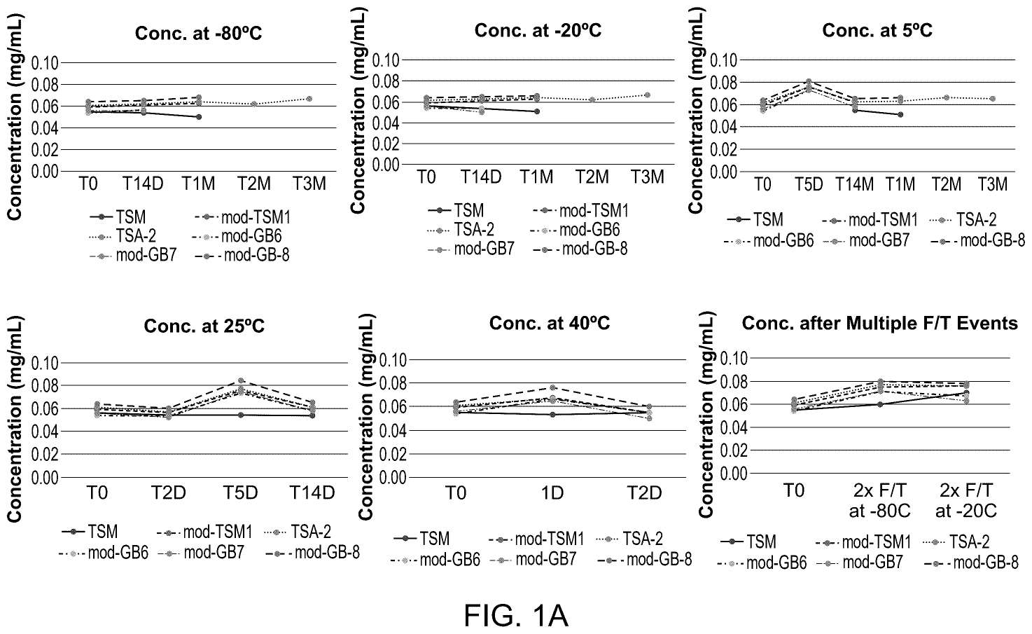

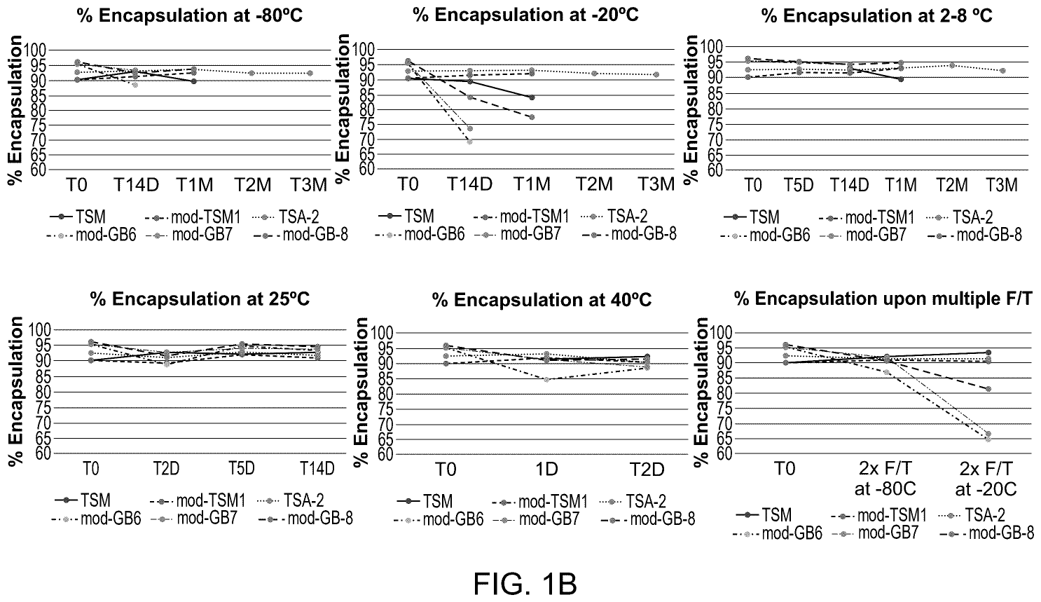

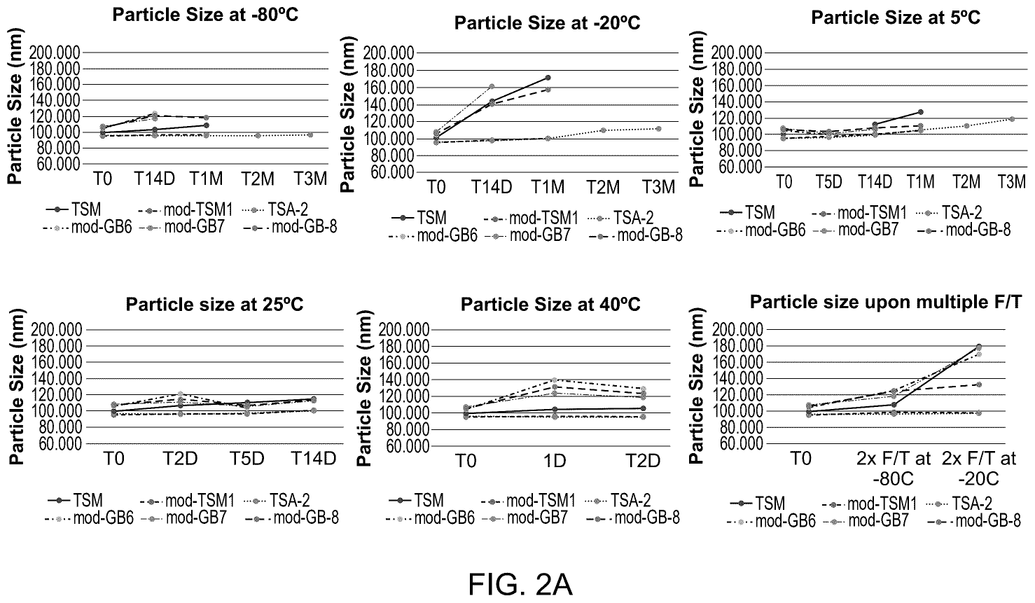

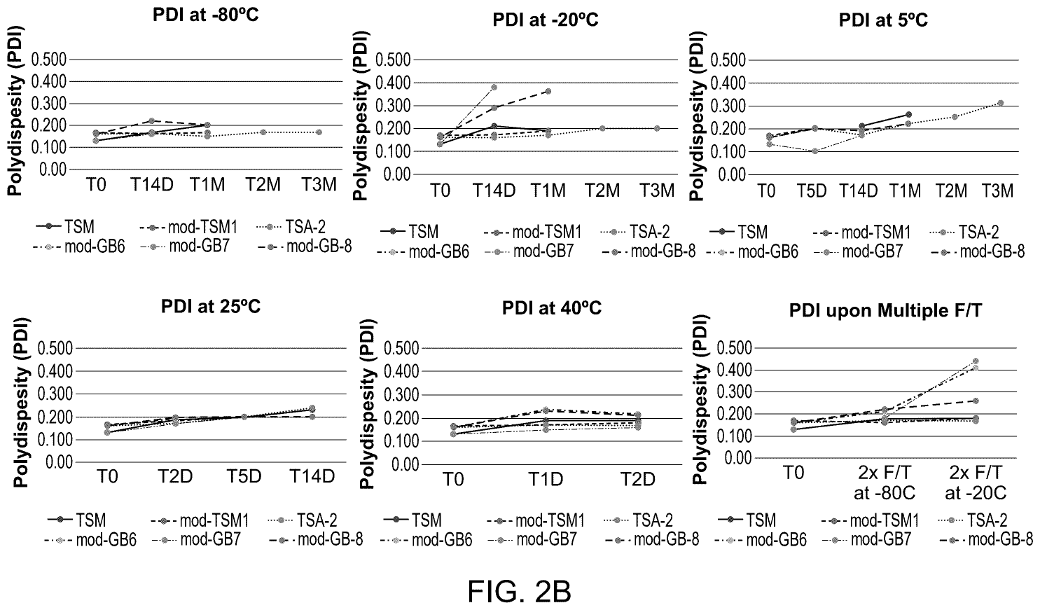

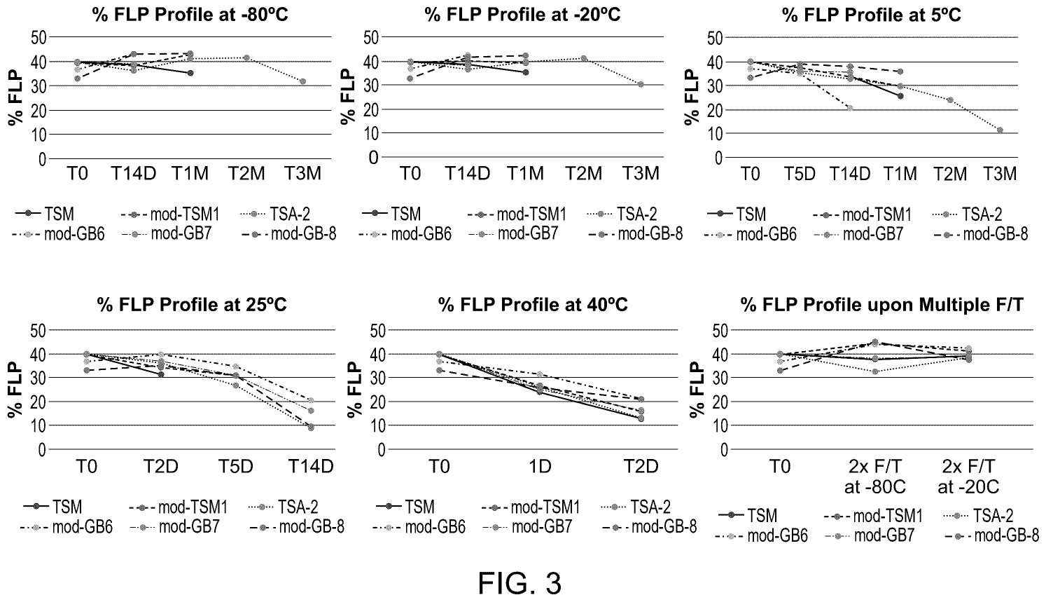

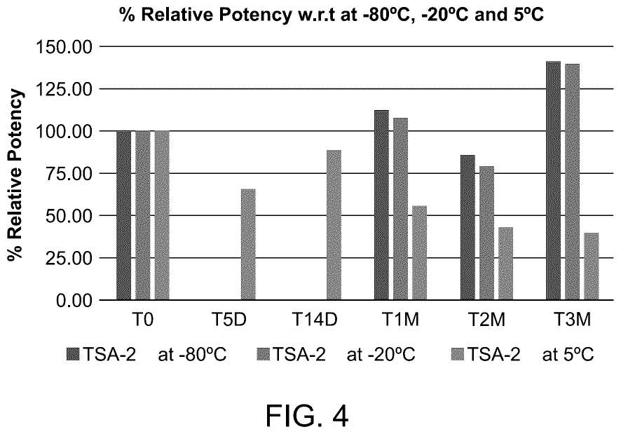

The comparison of samRNA-LNP stability of incubated samples under various temperature conditions including stressed condition and accelerated conditions for up to one month (1 M) indicated TSA-2 buffer to be the most promising data. TSA-2 formulation was further assessed for up to 3 months and as indicated by the data (FIG. 1-4) indicated stability at −80° C., −20° C. for up to 3 months, excellent stability upon multiple F/T events from either −80° C. or −20° C. storage conditions and up to 2 weeks stability under the refrigerated conditions of 5° C.

As indicated from the % encapsulation, DLS and cell-based potency data from the storage conditions at −80° C., −20° C., 5° C., and F/T data for TSA-2 buffer matrix, there was no increase in size, no drop in potency and no drop in % encapsulation efficiency for storage up to 3 months. Furthermore, at the refrigerated storage condition of 5° C., acceptable critical quality attributes are maintained for up to two (2) weeks. The data supports a marked improvement in DP stability in the investigational formulation matrix of TSA-2 over the existing TSM formulation matrix. In the current TSM formulation matrix, a marked drop in % encapsulation, and increase in size at the elevated storage condition of −20° C. is observed within 1 month of storage.

TSA-2 buffer was further analyzed in a second samRNA-LNP construct encoding a different cassette of approximately 6 kb in size for assessment of the robustness of the formulation matrix.

### Example 3: 3 Month Stability for samRNA-LNP DP Stability in Optimized TSA Formulation

Based on the results of TSA-2 buffer in the short-term stability study outlined above, a further optimization of excipient study was initiated.

In this short term stability study slight modifications to the amounts of the buffering agent Tris, stabilizing excipients Sucrose and Arginine and formulation pH were assessed. The compositions of the resulting investigational formulation matrix were as follows

| Reference | Formulation Matrix | pH |
|---|---|---|
| TSM | 5 mM Tris, 10 wt % Sucrose, 10 wt % Maltose | 7.8 |
| TSA-2 | 5 mM Tris, 15 wt % Sucrose, 50 mM Arg | 7.8 |
| TSA-3 | 5 mM Tris, 13 wt % Sucrose, 75 mM Arg | 7.8 |
| TSA-4 | 8 mM Tris, 13 wt % Sucrose, 75 mM Arg | 7.8 |
| TSA-5 | 5 mM Tris, 15 wt % Sucrose, 75 mM Arg | 7.5 |

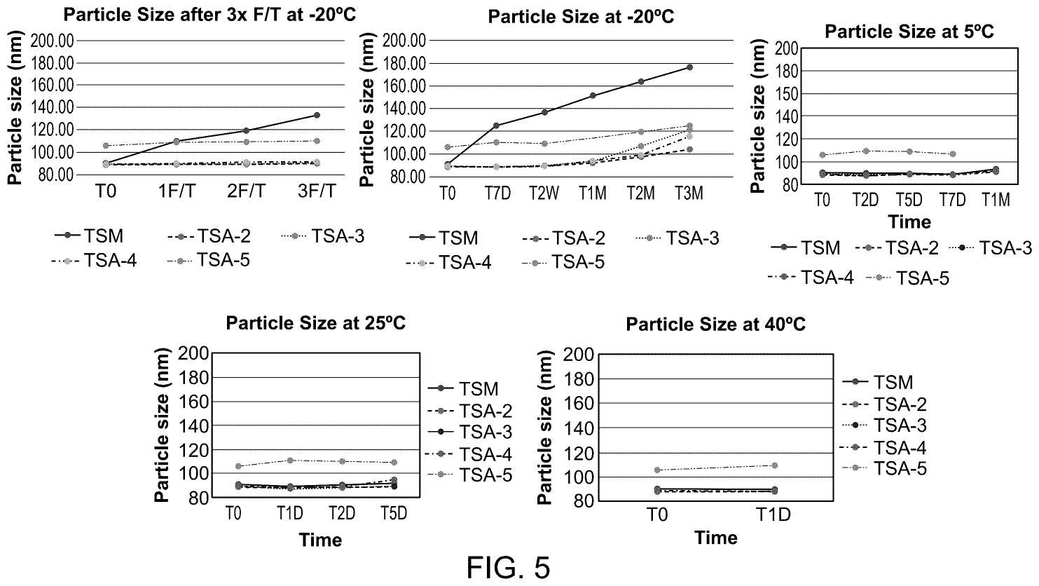

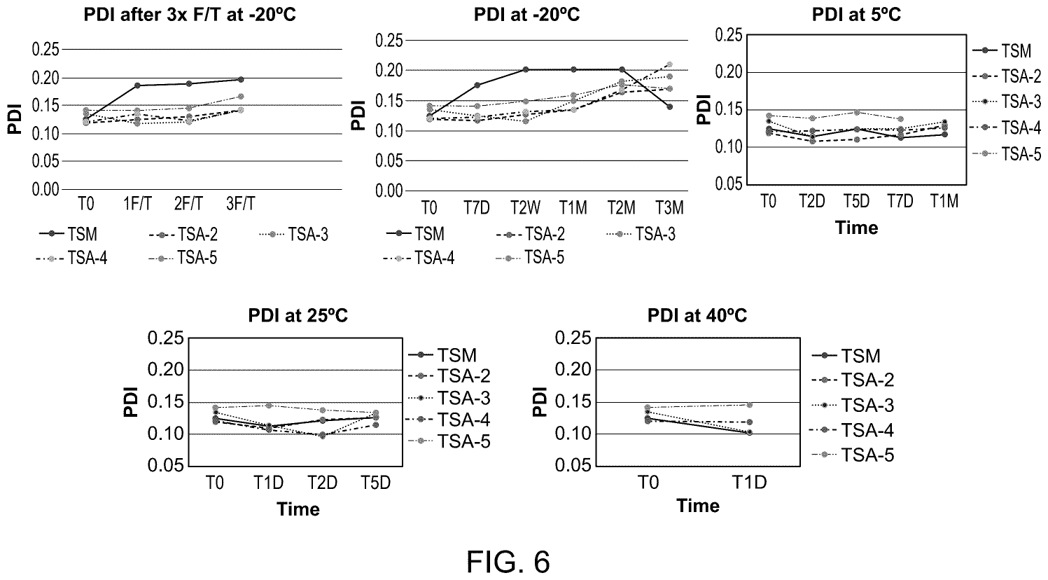

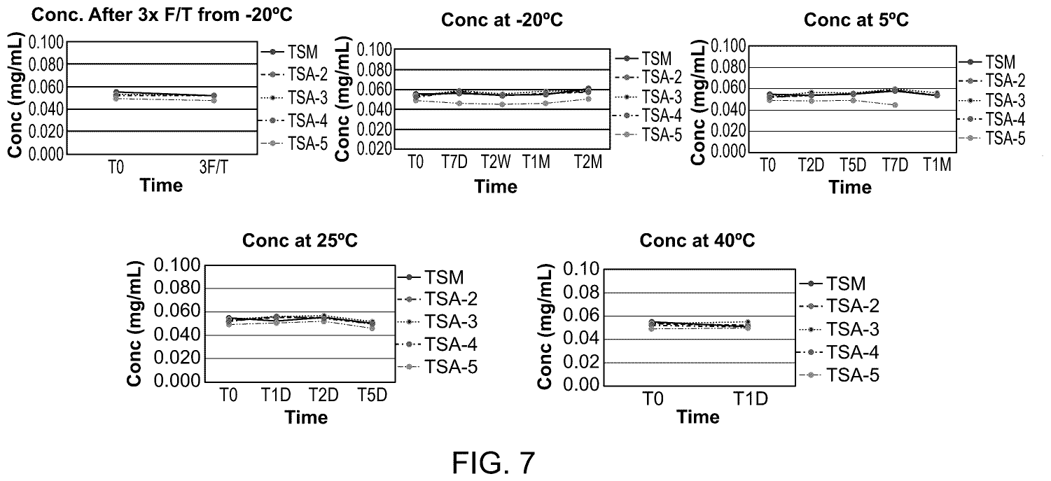

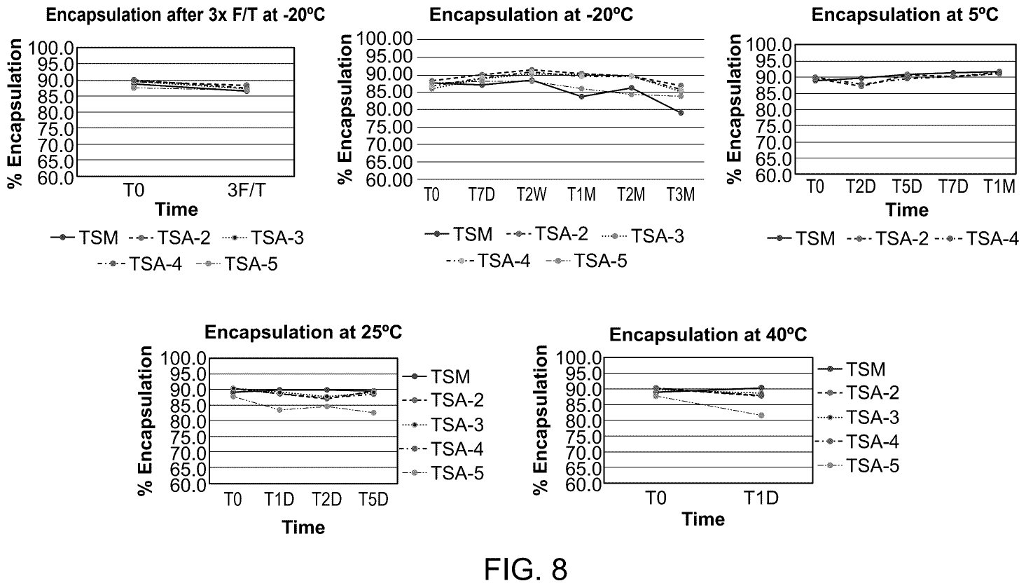

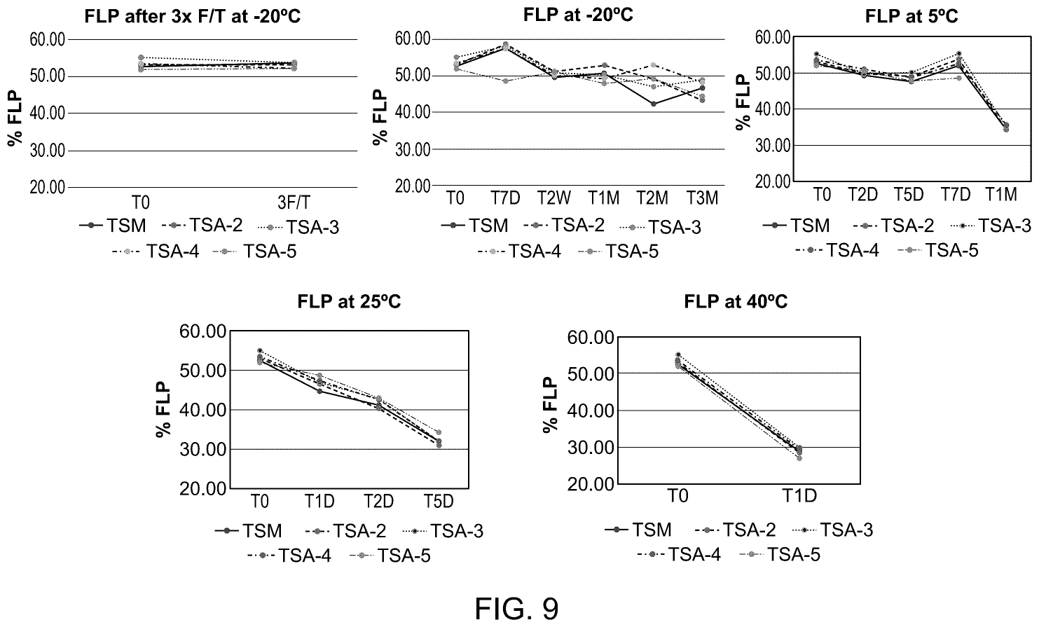

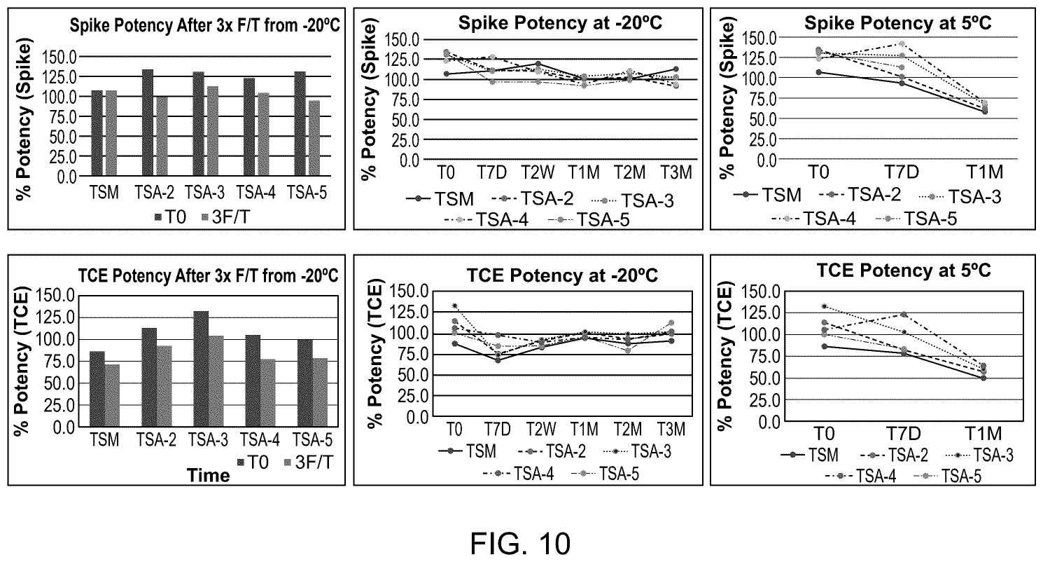

The aforementioned formulations were used in a short term stability study of the buffer exchanged samRNA-LNP DP at the incubation temperatures of −20° C., 5° C., 25° C. and 40° C. The formulations were assessed for stability trends by particle size and polydispersity determination, concentration and encapsulation determination, encapsulated RNA full length profile determination and cellular potency. The resulting data is captured in FIG. 5-10.

Effectiveness of the Tris Sucrose Arginine based formulation for DP was assessed via various critical product attributes (FIG. 5-FIG. 10). No appreciable change in CQAs were observed as a function of multiple freeze thaw (F/T) events from the intended storage temperature of −20° C. Furthermore, slight changes in buffering amounts, cryoprotectant amount and stabilizer amounts did not show appreciable differences in stability profile over 3 months storage at −20° C. Indeed, cell-based potency values were maintained above 50% for up to 3 Months at −20° C. and up to 1 month 5° C.

Thus, based on the generated data, TSA-4 formulation (8 mM Tris, 13 wt % Sucrose, 75 mM Arg) exhibited the most consistent stability profile as assessed by size, aggregation, concentration, encapsulation, full length samRNA-RNA profile, and cell-based potency during the stability assessment for up to 3 months at −80° C., −20° C., and up to 1 month at 5° C. when compared with TO (initial measurements). Accordingly, these data indicate that the optimized Tris-Sucrose-Arginine formulation, TSA-4 (8 mM Tris, 13 wt % Sucrose, 75 mM Arg), provides robust short-term samRNA-LNP stabilization at the intended storage condition of −20° C. Additionally, based on the trends of stability up to 3 months at −20° C., the expectation is TSA formulation enables long-term stability of samRNA-LNP DPs at −20° C.

Additionally, TSA-4 formulation (8 mM Tris, 13 wt % Sucrose, 75 mM Arg) was also evaluated as a formulation matrix to enable the generation of lyophilized samRNA-LNP DP that can enable long-term storage at 5° C. condition.

TSA-4 buffer and samRNA-LNP DP formulated in TSA-4 buffer were characterized for thermal properties (Tg′ and T_{C}) to help design a rational lyo cycle around the critical temperatures, Tg′ (Glass Transition temperature) and T_{c} (Lyophilized cake collapse temperature).

Lyo cycle development was initiated with the initial evaluation of Lyo compatibility assessment of the TSA-4 buffer by developing a base lyophilization cycle using TSA-4 buffer alone, followed by executing the lyo cycle for the samRNA-LNP product formulated in TSA-4 buffer.

Multiple interactions of lyophilized cycle optimization were performed (outlined in following table) to narrow down a robust lyophilized cycle that would enable short term stability of the DP in TSA formulation at the storage condition of 5° C.

| | |
|---|---|
| 1 | Evaluate various freezing temperatures and freezing rates in the |
| | Freezing step of Lyo Cycle |
| 2 | Optimize 1° drying shelf temperature |
| | Dependent on Tg′ and T_{c} form thermal properties of TSA-4 |
| | Important for elegant Lyophilized cake formation |
| 3 | Optimize 1° drying ramp rate |
| | Important for elegant Lyophilized cake formation |
| 4 | Optimize chamber pressure |
| | Important for effective vapor transport in 1° (primary dry) step of |
| | Lyo cycle |
| 5 | Optimize duration of primary drying |
| | Important for elegant Lyophilized cake and moisture content |
| 6 | Optimize 2° drying Temperature and rate |
| | Important for removing bound water and moisture content |
| 7 | Optimize duration of 2° drying |
| | Important for removing bound water and moisture content |

The final lyophilized DP stability study of DP formulated in TSA-4 buffer (2 mL of the DP at 0.06 mg/mL filled into 6 mL vials) was staged for short term stability study at the various temperature (−80° C., 5° C., 25° C. and 40° C.) and storage conditions for up to 3 months.

The data from the stability assessment of the lyophilized DP is captured in the following table.

| Storage Temp | | | Average | | | Potency | Potency |
|---|---|---|---|---|---|---|---|
| and Time | Size (nm) | PDI | FLP | Conc | Encapsulation | (TCE) | (Spike) |
| T0 | 81.8 | 0.18 | 61.83 | 0.050 | 74.5 | 128.61 | 116.06 |
| 2 W at 5° C. | 84.7 | 0.17 | 60.20 | 0.054 | 74.2 | 113.30 | 119.20 |
| 1 M at 5° C. | 85.7 | 0.16 | 60.20 | 0.050 | 71.4 | 75.55 | 68.28 |
| 2 M at 5° C. | 86.5 | 0.18 | 59.00 | 0.056 | 71.6 | 120.72 | 123.35 |
| 3 M at 5° C. | 81.1 | 0.169 | 53.40 | 0.058 | 78.50 | 117.67 | 108.9 |
| Ship study at 5° C. | 81.64 | 0.18 | 65.80 | 0.053 | 68.00 | 110.57 | 111.73 |
| 1 M at 25° C. | 86.1 | 0.15 | NT | 0.050 | 77.4 | 78.13 | 73.53 |
| 2 M at 25° C. | 84.45 | 0.16 | 6.10 | 0.057 | 79.30 | 42.34 | 41.57 |
| 3 M at 25° C. | 85.95 | 0.15 | 9.0 | 0.055 | 81.90 | 37.8 | 32.31 |
| 1 W at 40° C. | 88.8 | 0.15 | 5.74 | 0.500 | 78.9 | 90 | 103.4 |

Lyophilized DP exhibited elegant physical cake structure with no structural defects such as cracks, melt-back and micro-collapse.

Lyophilized samRNA-LNP DP also exhibited excellent DP quality for up to 3 months at 5° C. with no changes in any of the measured attributes including size, encapsulation, FLP and cellular potency

Additionally, the elegant cake allowed for acceptable reconstitution (reconstitution time within 2 minutes) without any foaming leading to the generation of corresponding liquid DP

Lyophilized DP was also evaluated for impact of shipping the DP at 5° C. Data generated from shipping study performed at 5° C. mimicked the observed stability profile (upon storage at 5° C. in the developmental lab setting) without shipping.

At the accelerated and stressed conditions of 25° C. and 40° C., data indicated a trend towards product degradation (loss in RNA full length profile) corroborated with a concurrent drop in cellular potency with increase in duration.

However, the current data still supports acceptable DP attributes for up to 1 month at 25° C. to enable accidental exposure or product excursions at clinical sites from recommended 5° C.

Taken together, the stability data generated from the DP in TSA-4 buffer indicates robust stability for up to 3 months at 5° C. and potential for long term stability up to 2 years at the storage condition of 5° C.

1. A pharmaceutical composition comprising an RNA-based expression system, further comprising a buffer, an amino acid, and a cryoprotectant.

2. The pharmaceutical composition of claim 1, wherein the RNA-based expression system is selected from the group consisting of a messenger RNA (mRNA)-based expression system, a circular (circRNA)-based expression system, single guide RNA (sgRNA)-based expression system, and a self-amplifying RNA (samRNA) expression system.

3. The pharmaceutical composition of claim 1, wherein the RNA-based expression system is a self-amplifying RNA (samRNA) expression system.

4. The pharmaceutical composition of any of claims 1-2, wherein the amino acid is selected from histidine, lysine, arginine, glutamine, and arginine or a pharmaceutically acceptable salt thereof.

5. The pharmaceutical composition of claim 6, wherein the amino acid is arginine.

6. The pharmaceutical composition of any of claims 1-5, wherein the amino acid has a concentration of 10-140 mM.

7. The pharmaceutical composition of any of claims 1-5, wherein the amino acid has a concentration of 25-125 mM.

8. The pharmaceutical composition of any of claims 1-5, wherein the amino acid has a concentration of 50-100 mM.

9. The pharmaceutical composition of any of claims 1-5, wherein the amino acid has a concentration of 70-80 mM.

10. The pharmaceutical composition of any of claims 1-5, wherein the amino acid has a concentration of about 75 mM.

11. The pharmaceutical composition of claims 1-10, wherein the composition has a pH of 6.5-9.1.

12. The pharmaceutical composition of claim 11, wherein the pH is 7.3-8.3.

13. The pharmaceutical composition of claim 11, wherein the pH is about 7.8.

14. The pharmaceutical composition of any of claims 1-13, wherein the buffer is selected from the group consisting of a buffer is selected from the group consisting of citrate, succinate, malate, phosphate, histidine, glycine, MOPS, HEPES, Tris, and Bis-Tris.

15. The pharmaceutical composition of claim 14, wherein the buffer is Tris.

16. The pharmaceutical composition of claim 14, wherein the buffer is not a phosphate buffer.

17. The pharmaceutical composition of any of claims 1-15, wherein the buffer has a concentration of 1-15 mM.

18. The pharmaceutical composition of any of claims 1-15, wherein the buffer has a concentration of 3-12 mM.

19. The pharmaceutical composition of any of claims 1-15, wherein the buffer has a concentration of 6-10 mM.

20. The pharmaceutical composition of any of claims 1-15, wherein the buffer has a concentration of about 8.0 mM.

21. The pharmaceutical composition of any of claims 1-15, wherein the buffer has a concentration greater than 5.0 mM

22. The pharmaceutical composition of any of claims 1-20, wherein the pharmaceutical composition comprises a cryoprotectant.

23. The pharmaceutical composition of claim 22, wherein the cryoprotectant is selected from the group consisting of ethanol, sucrose, maltose, lactose, glucose, galactose, trehalose, raffinose, other polyols and polyhydric alcohols.

24. The pharmaceutical composition of any of claims 22-23, wherein the cryoprotectant is 6-18 wt % of the pharmaceutical composition.

25. The pharmaceutical composition of any of claims 22-23, wherein the cryoprotectant is 11-15 wt % of the pharmaceutical composition.

26. The pharmaceutical composition of any of claims 22-23, wherein the cryoprotectant is about 13 wt % of the pharmaceutical composition.

27. The pharmaceutical composition of claims 1-26, wherein the cryoprotectant is sucrose.

28. A pharmaceutical composition comprising a samRNA-based expression system, and further comprising
70-80 mM arginine;
11-15 wt % Sucrose; and
6-10 mM Tris;
wherein the pharmaceutical composition has a pH of 7.6-8.0.

29. A pharmaceutical composition comprising a samRNA-based expression system, and further comprising
about 75 mM arginine;
about 13 wt % Sucrose; and
about 8 mM Tris;
wherein the pharmaceutical composition has a pH of about 7.8.

30. The pharmaceutical composition of any of claims 1-29, wherein stability of the pharmaceutical composition remains at a temperature of at least −20° C., at least 5° C., or at least 25° C.

31. The pharmaceutical composition of claim 30, wherein the stability is assessed by one or more assays comprising particle size, PDI, samRNA concentration, percent encapsulation, Full Length Profile (FLP) of samRNA and potency.

32. The pharmaceutical composition of claim 31, wherein the potency of the RNA-based expression system of the pharmaceutical composition is above about 40% of potency after storage as compared to potency before storage.

33. The pharmaceutical composition of claim 32, wherein the pharmaceutical composition is stored at about −20° C.

34. The pharmaceutical composition of claim 32, wherein the pharmaceutical composition is stored at about 5° C.

35. The pharmaceutical composition of claim 32, wherein the pharmaceutical composition is stored at about 25° C.

36. The pharmaceutical composition of any of claims 32-35, wherein the pharmaceutical composition is stored for at least 1 day.

37. The pharmaceutical composition of any of claims 32-35, wherein the pharmaceutical composition is stored for at least 3 days.

38. The pharmaceutical composition of any of claims 32-35, wherein the pharmaceutical composition is stored for at least 5 days.

39. The pharmaceutical composition of any of claims 32-35, wherein the pharmaceutical composition is stored for at least 1 week.

40. The pharmaceutical composition of any of claims 32-35, wherein the pharmaceutical composition is stored for at least 2 weeks.

41. The pharmaceutical composition of any of claims 32-35, wherein the pharmaceutical composition is stored for at least 1 month.

42. The pharmaceutical composition of any of claims 32-35, wherein the pharmaceutical composition is stored for at least 3 months.

43. The pharmaceutical composition of any of claims 32-35, wherein the pharmaceutical composition is stored for at least 6 months.

44. The pharmaceutical composition of any of claims 1-43, wherein the buffer is not a phosphate buffer.

45. The pharmaceutical composition of any of claims 1-43, wherein the pharmaceutical composition does not comprise a phosphate buffer.

46. The pharmaceutical composition of any of claims 1-45, wherein the pharmaceutical composition is in a liquid formulation.

47. The pharmaceutical composition of any of claims 1-45, wherein the pharmaceutical composition is in a lyophilized formulation.

48. The pharmaceutical composition of any of claim 47, wherein water content of the pharmaceutical composition is <5 wt %.

49. The pharmaceutical composition of any of claims 1-48, wherein the pharmaceutical composition is stored in a container comprising SiO₂.

50. A method for inducing an immune response in a subject, the method comprising administering to the subject the composition of claims 1-48.

51. The method of claim 50, wherein the composition is administered intramuscularly (IM), intradermally (ID), subcutaneously (SC), or intravenously (IV).

52. The method of claim 51, wherein the composition is administered intramuscularly.

53. The method of any of claims 50-52, the method further comprising administration of one or more immune modulators, optionally wherein the immune modulator is administered before, concurrently with, or after administration of the composition or pharmaceutical composition.

54. The method of claim 53, wherein the one or more immune modulators are selected from the group consisting of: an anti-CTLA4 antibody or an antigen-binding fragment thereof, an anti-PD-1 antibody or an antigen-binding fragment thereof, an anti-PD-L1 antibody or an antigen-binding fragment thereof, an anti-4-1BB antibody or an antigen-binding fragment thereof, or an anti-OX-40 antibody or an antigen-binding fragment thereof.

55. The method of any of claim 53 or 54, wherein the immune modulator is administered intravenously (IV), intramuscularly (IM), intradermally (ID), or subcutaneously (SC).

56. The method of claim 55, wherein the subcutaneous administration is near the site of the composition or pharmaceutical composition administration or in close proximity to one or more vector or composition draining lymph nodes.

57. The method any of claims 49-56, further comprising administering to the subject a second vaccine composition.

58. The method of claim 57, wherein the second vaccine composition is administered prior to the administration of the composition of any of claims 1-48.

59. The method of claim 57, wherein the second vaccine composition is administered subsequent to the administration of the composition of any of claims 1-48.

60. The method of any of claims 57-59, wherein the second vaccine composition is the same as the composition of any of claims 1-48.

61. The method of any of claims 57-59, wherein the second vaccine composition is different from the composition any of claims 1-48.

62. A method of preparing a pharmaceutical composition of any of claims 1-48, comprising the step of lyophilization.

63. The method of claim 62, wherein the step of lyophilization comprises a secondary drying step.

64. The method of claim 63, wherein conditions for the secondary drying step comprise a shelf temperature of about 10° C.

65. The method of claim 63, wherein conditions for the secondary drying step comprise a shelf temperature between 5-15° C.

66. The method of any of claims 62-65, wherein conditions for the secondary drying step comprise a vacuum of about 50 mTorr.

67. The method of any of claims 62-65, wherein conditions for the secondary drying step comprise a vacuum between 25-75 mTorr.

68. The method of claim 63, wherein conditions for the secondary drying step comprise
a) a shelf temperature of about 10° C.;
b) a vacuum of about 50 mTorr;
c) a ramp rate of about 0.1° C./min; and
d) a duration of about 1040 min.

69. The method of any of claims 63-68, wherein the step of lyophilization comprises a primary drying step.

70. The method of claim 69, wherein conditions for the primary drying step comprise a shelf temperature of about −37° C.

71. The method of claim 69, wherein conditions for the primary drying step comprise a shelf temperature between −62 to −12° C.

72. The method of any of claims 69-71, wherein conditions for the primary drying step comprise a vacuum of about 50 mTorr.

73. The method of any of claims 69-71, wherein conditions for the primary drying step comprise a vacuum between 25-75 mTorr.

74. The method of any of claims 63-73, wherein the step of lyophilization comprises a freezing step.

75. The method of claim 74, wherein conditions for the freezing step comprise a shelf temperature of about −55° C.

76. The method of claim 74, wherein conditions for the freezing step comprise a shelf temperature between −60 to −35° C.

77. The method of claim 69, wherein conditions for the primary drying step comprise
a) a shelf temperature of about −37° C.;
b) a vacuum of about 50 mTorr;
c) a ramp rate of about 0.5° C./min; and
d) a duration of about 4800 min.

78. A composition prepared by the method of any of claims 63-77.


SEQUENCE LISTING (9 sequences)

SEQ ID NO: 1 - protein, 5 aa, synthetic construct
GPGPG

SEQ ID NO: 2 - protein, 100 aa, synthetic construct
VTNTEMFVTAPDNLGYMYEVQWPGQTQPQIANCSVYDFFVWLHYYSVRDTVTNTEMFVTAPDNLGYMYEVQWPGQTQPQIANCSVYDFFVWLHYYSVRDT

SEQ ID NO: 3 - protein, 25 aa, synthetic construct
VTNTEMFVTAPDNLGYMYEVQWPGQ

SEQ ID NO: 4 - protein, 25 aa, synthetic construct
TQPQIANCSVYDFFVWLHYYSVRDT

SEQ ID NO: 5 - DNA, 19 bp, synthetic construct
  source 1..19: mol_type = other DNA
taatacgactcactatagg

SEQ ID NO: 6 - DNA, 20 bp, synthetic construct
  source 1..20: mol_type = other DNA
taatacgactcactataggn

SEQ ID NO: 7 - DNA, 17 bp, synthetic construct
  source 1..17: mol_type = other DNA
taatacgactcactata

SEQ ID NO: 8 - DNA, 18 bp, synthetic construct
  source 1..18: mol_type = other DNA
taatacgactcactatan

SEQ ID NO: 9 - DNA, 17 bp, synthetic construct
  source 1..17: mol_type = other DNA
atttaggtgacactata